(19) United States
(12) Patent Application Publication
(10) Pub. No.: US 2006/0200013 A1
(43) Pub. Date: Sep. 7, 2006

(54) Systems and methods for maintaining optical fixation and alignment

(76) Inventors: John L. Smith, Fairplay, CA (US); Robert G. Messerschmidt, Santa Clara, CA (US); Mark J. Rice, Jacksonville, FL (US); Raymond T. Hebert, Florence, OR (US); Todd D. Lorey, Santa Cruz, CA (US); John Lemke, Pleasanton, CA (US)

Correspondence Address: WILSON SONSINI GOODRICH & ROSATI, 650 PAGE MILL ROAD, PALO ALTO, CA, 94304-1050, US

(21) Appl. No.: 11/219,334

(22) Filed: Sep. 2, 2005

Related U.S. Application Data
(63) Continuation-in-part of application No. 11/005,767, filed on Dec. 6, 2004.
Application No. 11/005,767 is a continuation of application No. 10/642,104, filed on Aug. 15, 2003, now Pat. No. 6,889,069.
Application No. 10/642,104 is a continuation of application No. 10/012,902, filed on Oct. 22, 2001, now Pat. No. 6,650,915.
(63) Continuation-in-part of application No. 10/863,619, filed on Jun. 8, 2004.
(60) Provisional application No. 60/608,036, filed on Sep. 7, 2004.

(51) Int. Cl. A61B 5/00

(52) U.S. Cl. 600/319; 600/316

(57) ABSTRACT

Blood glucose levels in an individual are determined by aligning a region of the retina of the eye of an individual with an instrument, measuring a regeneration rate of the retinal visual pigment of the individual, and using the measured regeneration rate to calculate the blood glucose level. The apparatus for measuring the blood glucose comprises a blood glucose analysis instrument adapted to determine blood glucose concentration from information regarding regeneration rate of the visual pigment in the retina region of an eye, and an alignment mechanism adapted to align the retina region with at least a portion of the device.

### CROSS-REFERENCE

This application is a continuation-in-part application of Ser. No. 11/005,767, filed Dec. 6, 2004, which is a continuation of Ser. No. 10/642,104, filed Aug. 15, 2003, now U.S. Pat. No. 6,889,069, which is a continuation of Ser. No. 10/012,902, filed Oct. 22, 2001, now U.S. Pat. No. 6,650,915; this application is also a continuation-in-part of Ser. No. 10/863,619, filed Jun. 8, 2004. These applications are incorporated herein by reference in their entirety, and we claim priority to these applications under 35 USC § 120. This application also claims the benefit of U.S. Provisional Application No. 60/608,036, filed Sep. 7, 2004, which is incorporated herein by reference in its entirety.

### BACKGROUND OF THE INVENTION

Because of the importance of blood glucose measurements in managing and treating diabetes, many blood glucose measurement systems have been described in the art. The pain and inconvenience of traditional approaches for monitoring glucose, which involve puncturing the skin to obtain a drop of blood, have resulted in many suggestions for methods of measuring glucose without causing pain or discomfort. These approaches are generally referred to as “noninvasive.”

Because of the optical transparency of the eye, many of the approaches described in the art measure glucose within the eye using light of various wavelengths, polarizations and orientations. For example, March U.S. Pat. No. 3,958,560 and Quandt U.S. Pat. No. 3,963,019 describe attempts to measure glucose in the aqueous humor contained in the anterior chamber of the eye (the fluid-filled space between the cornea and the iris). Other eye-based glucose monitors are described in Ansari U.S. Pat. No. 6,704,588; Lambert U.S. Pat. No. 6,181,957; Stark U.S. Pat. No. 5,433,197; Rice U.S. Pat. No. 6,305,804; and Rice U.S. Pat. No. 6,477,394.

The need for gaze direction or fixation during a measurement or other procedure has been addressed with differing degrees of sophistication, depending on the application or measurement. For example, when a physician examines a patient's eye with an ophthalmoscope, the patient may simply be asked to direct his gaze to a corner of the examining room. Measurement of inter-pupillary distance requires a gaze fixation point, as shown in the product and described in the user's guide for the Digital PD Meter, Reichert Ophthalmic Instruments, 3374 Walden Ave, Depew, N.Y. 14043. When a retinal photograph is made, the patient's gaze direction may be maintained by fixation points or other indicia, as disclosed in U.S. Pat. No. 5,120,122. When retinal patterns are scanned for identification or other biometric applications, the gaze may be fixated or actively tracked as disclosed in U.S. Pat. No. 6,120,461.

Earlier attempts to measure glucose levels in the eye have also required that the patient's gaze be directed toward a selected image or target, as disclosed in Stark U.S. Pat. No. 5,433,197, which also discloses using a blinking light to direct the gaze, as does Lambert U.S. Pat. No. 6,181,957. Additionally, Ansari U.S. Pat. No. 6,704,588 also describes the use of a fixating light as an alignment guide for determination of glucose levels in the eye. In all three of these disclosures, however, the measurement site for the glucose measurement in these is not the retina but the anterior chamber of the eye, and the gaze fixation is used to locate that portion of the eye in relation to a beam of light, either polarized light to detect changes in optical rotation in the first two cases, or laser light for examination of the fluid in the anterior chamber by Raman spectroscopy in the third case.

Castano U.S. Pat. No. 5,713,353, also discloses a fixation light or pattern as part of a psychophysical or visual method for glucose estimation based on interpretation of patterns displayed to the patient, with differences in pattern recognition ascribed to relative perception through two separate retinal visual processing systems.

What is needed are better and more accurate glucose monitoring systems. In addition, in order to allow accurate optical measurements of glucose within the eye, what is also needed are devices and techniques to assist or improve the ability of a patient's eye and head to maintain fixation in a desired location relative to the system and without excessive movement for the duration of an examination or test.

### SUMMARY OF THE INVENTION

The present invention carries out measurements of blood glucose in a repeatable, non-invasive manner by measurement of the rate of consumption of glucose, or the rate of production of another substance which is dependent on the glucose concentration of the individual, as an indication of the individual's glucose concentration. The devices and methods of this invention also improve the ability of a patient's eye and head to maintain fixed in a desired location relative to the system and without excessive movement for the duration of a glucose test. The rate of consumption of glucose (or the rate of production of a second glucose concentration-dependent substance) can be the result of the consumption of glucose by a specific organ or part of the body, or by a specific biochemical process in the body. One such process is the rate of regeneration of retinal visual pigments, such as cone visual pigments. The rate of regeneration of visual pigments is dependent upon the blood glucose concentration, by virtue of the glucose concentration limiting the rate of production of a cofactor, NADPH, which is utilized in the rate-determining step of the regeneration of visual pigments. Thus, by measuring the visual pigment regeneration rate, blood glucose can be accurately determined. One embodiment of this invention exposes the retina to light of selected wavelengths at selected times and analyzes the reflection (as color or darkness) from a selected portion of the exposed region of the retina, preferably from the foveal region of the retina.

In one aspect of the invention, the blood glucose is determined by aligning a region of the retina of the eye of an individual with an instrument, measuring a regeneration rate of the retinal visual pigment of the individual, and using the measured regeneration rate to calculate the blood glucose level. The apparatus for measuring the blood glucose comprises a blood glucose analysis instrument adapted to determine blood glucose concentration from information regarding regeneration rate of the visual pigment in the retina region of an eye, and an alignment mechanism adapted to align the retina region with at least a portion of the device.

In another aspect of the invention, blood glucose concentration is measured by maintaining alignment of a retina region of the eye of the patient with respect to a measurement instrument for a measurement period, taking first and second measurements from a portion of the eye with the measurement instrument at first and second points in the measurement period and using the first and second measurements to calculate the blood glucose concentration.

### INCORPORATION BY REFERENCE

All publications and patent applications mentioned in this specification are herein incorporated by reference to the same extent as if each individual publication or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 is a general diagram of an exemplary embodiment of a system for non-invasive measurement of blood glucose using retinal visual pigment.

FIG. 2 is a schematic diagram of an apparatus for measurement of blood glucose in accordance with an exemplary embodiment.

FIG. 3A is a representation of a pair of goggles, illustrating a potential form factor of an exemplary embodiment.

FIG. 3B is a representation of a hand-held monocular device, illustrating a potential form factor of an exemplary embodiment.

FIG. 3C is a representation of a hand-held binocular device, illustrating a potential form factor of an exemplary embodiment.

FIG. 3D is a representation of a head-mounted device, illustrating a potential form factor of an exemplary embodiment.

FIG. 4 is a diagram illustrating the effect of applying pulses of illuminating light to cause bleaching of visual pigments followed by pulses of lower intensity light to allow imaging and determination of the rate of regeneration of the visual pigments.

FIG. 5 is a schematic diagram of a further optical illumination and detection system that may be utilized in the apparatus of FIGS. 1 and 2.

FIG. 6 is a schematic diagram of an optical illumination and detection system that may be utilized in the apparatus of FIGS. 1 and 2.

FIG. 7 is a graph of an example reflectance trace.

FIG. 8 is an expanded view of a portion of the graph of FIG. 7 showing a trace where the subject has a relatively high glucose level.

FIG. 9 is a closer view of a portion of a reflectance trace graph where a subject has a low glucose level.

FIG. 10 is a depiction of two graphs having a linear portion of regeneration data near the beginning of a post-bleach phase, the top graph from a patient with a low glucose and the bottom graph from a patient with a high glucose;

FIG. 11 is a depiction of the “steady-state” method of glucose measurement used in the apparatus of FIG. 6.

FIG. 12 is a graph of glucose readings using the apparatus of FIG. 6 compared to glucose readings using a finger stick blood glucose measurement.

FIG. 13 is a Clarke Error Grid with measured and referenced glucose measurements using the apparatus of FIG. 6.

FIG. 14 illustrates a generic optical measurement system.

FIG. 15 illustrates one embodiment of an arrangement of components in an integrated optical system.

FIG. 16 illustrates another embodiment of an exemplary integrated optical system.

FIG. 17 illustrates a fixation object embodiment as viewed by a subject.

FIG. 18A-H illustrate a series of time elapsed eyepiece images.

FIG. 18I illustrates a time elapsed image of a fixation object as it moves in the direction of the arrow.

FIG. 19 A-B illustrates the change in reflected light via different images.

FIG. 20A-I illustrates a series of elapsed eyepiece images.

FIG. 21 illustrates an exemplary system designed to allow the detection and correction of head movement.

FIG. 22A-B illustrates appearances of the overall image as seen by the user;

FIG. 23A-B illustrates other appearances of the overall image as seen by the user.

FIG. 24A-B illustrates various motions between the eye and the beam of light.

### DETAILED DESCRIPTION OF THE INVENTION

Rhodopsin is the visual pigment contained in the rods (that allow for dim vision) and cone visual pigments sometimes termed “cone opsins”) that allow for central and color vision are contained in the cones of the retina. The outer segments of the rods and cones contain large amounts of visual pigment, stacked in layers lying perpendicular to the light incoming through the pupil. As visual pigment absorbs light, it breaks down into intermediate colorless molecular forms and initiates a signal that proceeds down a tract of nerve tissue to the brain, allowing for the sensation of sight. During normal vision this bleaching process occurs continuously. Light that reacts with the visual pigments causes a breakdown of those pigments. This phenomenon is termed bleaching, since the retinal tissue loses its color content when light is directed onto it. In addition, regeneration of the visual pigments occurs at all times, even during the bleaching process. Rod visual pigment absorbs light energy in a broad band centered at 500 nm, whereas the three different cone visual pigments or opsins have broad overlapping absorption bands peaking at approximately 430, 550, and 585 nm, which correspond to blue, green, and red cones, respectively.

The rods and cones of the retina are arranged in specific locations in the back of the eye. The cones, which provide central and color vision, are located with their greatest density in the area of the fovea centralis in the retina. The fovea covers a circular area with a diameter of about 1.5 mm. The rods are found predominately in the more peripheral portions of the retina and contribute to vision in dim light.

Visual pigment consists of 11-cis-retinal and a carrier protein, which is tightly bound in the outer segments of the cones and rods. 11-cis-retinal is the photoreactive portion of visual pigment, which is converted to all-trans-retinal when a photon of light in the active absorption band strikes the molecule. This process goes through a sequence of chemical reactions (called visual pigment regeneration), including all-trans-retinal isomerizing back to 11-cis-retinal. During the initial portion of this series of chemical steps, the nerve fiber, which is attached to that particular rod or cone, undergoes a stimulus that is perceived in the brain as a visual signal. During this process, an electrical signal is generated that can be measured on an electroretinogram (ERG) or electroencephalogram (EEG).

Following the conversion of 11-cis-retinal to all-trans-retinal, the 11-cis-retinal is regenerated by a series of steps that result in 11-cis-retinal being recombined with an opsin protein in the cell or disk membrane. A critical (and rate-limiting) step in this regeneration pathway is the reduction of all-trans-retinal to all-trans-retinol using the enzyme all-trans-retinol dehydrogenase (ATRD), which requires NADPH as the direct reduction energy source. In a series of experiments, Futterman et al. have proven that glucose, via the pentose phosphate shunt (PPS), provides virtually all of the energy required to generate the NADPH needed for this critical reaction. S. Futterman, et al., “Metabolism of Glucose and Reduction of Retinaldehyde Retinal Receptors,” J. Neurochemistry, 1970, 17, pp. 149-156. Without glucose or its immediate metabolites, only very small amounts of NADPH are formed and visual pigment cannot regenerate.

In addition, Ostroy, et al. have shown that the extracellular glucose concentration has a major effect on visual pigment regeneration. S. E. Ostroy, et al., “Extracellular Glucose Dependence of Rhodopsin Regeneration in the Excised Mouse Eye,” Exp. Eye Research, 1992, 55, pp. 419-423. Since glucose is the primary energy source for visual pigment regeneration, embodiments of the present invention utilize this relationship to measure blood glucose concentrations.

According to embodiments of the present invention, light may be used to break down (or bleach) the visual pigment, and reflected light from the retina can be subsequently analyzed over a period of time to determine the regeneration rate of the visual pigment. Measurement of an unknown blood glucose concentration is accomplished by development of a relationship between the reflected light data (indicating the visual pigment regeneration rate) and corresponding clinically determined blood glucose concentration values. With either imaging or non-imaging embodiments of this invention, a steady-state illuminating light or a varying illuminating light may be applied to induce bleaching and a steady-state illuminating light or a varying illuminating light may be applied to determine the regeneration rate of the visual pigment. Measurement of regeneration rate may also be accomplished during the bleaching phase, as regeneration of the visual pigments occurs even while the pigments are being bleached. In addition, measurement of visual pigment regeneration may be made without a formal bleaching event. The device can be preferably used by the patient in a self-testing mode, or the device may be used by an operator. Pulsed or other light-varying techniques may be used to measure the regeneration rate of the visual pigment.

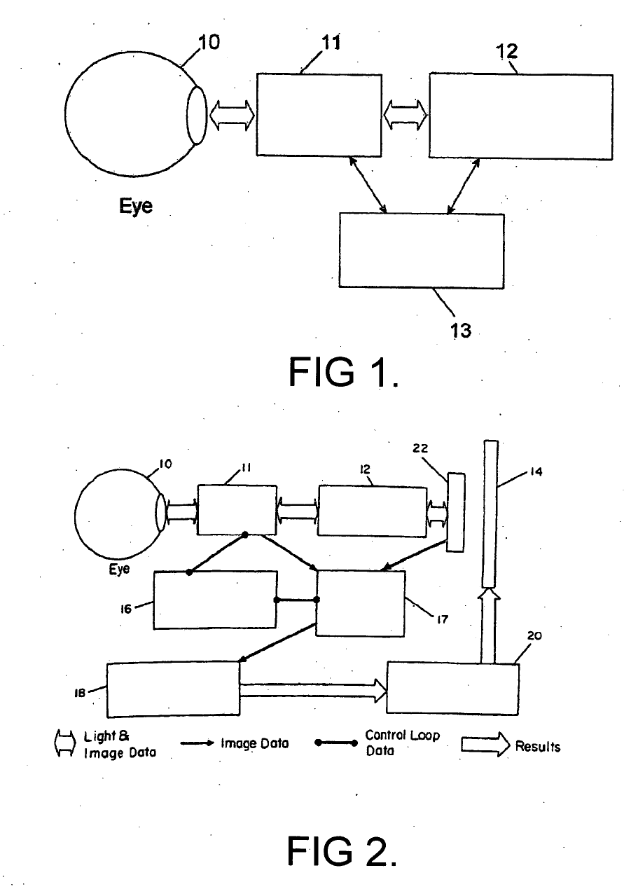

Under some methods of determining blood glucose concentration, the subject's eye is illuminated for a period of time, and light reflected from the retina is detected and analyzed. For example, FIG. 1 illustrates one embodiment of the present invention. The eye of the patient is illustrated at 10, with the optical system for directing light into the eye and obtaining light emitted from the eye shown as 11. The illumination system 12 contains elements for directing light through the pupil and onto the retina for the breakdown of visual pigment regeneration (bleaching). The data capture and analysis system 13 has elements for the measurement of the reflected light, calculation of the visual pigment regeneration rate, and conversion of this information into the blood glucose value.

With either imaging or non-imaging embodiments of this invention, light may be used to break down (or bleach) the visual pigment. Reflected light from the retina can be subsequently analyzed over a period of time to determine the regeneration rate of the visual pigment. Measurement of an unknown blood glucose concentration is accomplished by development of a relationship between the reflected light data (indicating the visual pigment regeneration rate) and corresponding clinically determined blood glucose concentration values. With either imaging or non-imaging embodiments of this invention, a steady-state illuminating light or a varying illuminating light may be applied to induce bleaching and a steady-state illuminating light or a varying illuminating light may be applied to determine the regeneration rate of the visual pigment.

Measurement of regeneration rate may also be accomplished during the bleaching phase; regeneration of the visual pigments occurs even while the pigments are being bleached. In addition, measurement of visual pigment regeneration may be made without a formal bleaching event. Pulsed or other light-varying techniques may be used to measure the regeneration rate of the visual pigment. While the device can be used by the patient in a self-testing mode, the device may be used by an operator to test the patient.

FIG. 2 illustrates an embodiment of the present invention using imaging. In this embodiment, the illumination system 12 provides illuminating light to image the retina. The illumination system 12 may be a monochromatic or multiple discrete wavelength light source that provides light for imaging the retina. In some embodiments, the system provides light for imaging coaxially to reduce the likelihood of extraneous reflections from the interior or exterior of the eye. The light from the illumination system is projected through the pupil, using optics system 11. The wavelength of this light source is selected dependent upon the particular visual pigment to be analyzed. Although any visual wavelength of light could be used, the light intended for absorption by visual cone pigments could be centered at 540 nm for green cones and 585 nm for red cones. Alternatively, other wavelengths which maximize either sensitivity, contrast or signal-to-noise ratio of the measurement may be utilized, and wavelengths may be selected to avoid interference with light of other wavelengths used for fixation or alignment. Illumination light may be composed of two or more separate lighting systems, such as a xenon strobe, multiple laser diodes, or light-emitting diodes (LEDs).

Infrared imaging (using, e.g., a filtered halogen or laser diode source) may be utilized to align the retina prior to imaging in the visual wavelengths. The light is reflected from the retina of the eye 10 and passed through the pupil opening of the eye to the optics system 11 and through the illumination system 12 entering, e.g., a charge coupled device (CCD) or complementary metal-oxide semiconductor (CMOS) image detector 22. The illumination system 12 and optics system 11 may be similar to systems used in existing non-mydriatic findus cameras.

In another embodiment, viewing system 14, for example, a liquid crystal display (LCD) screen, may receive the image data and display the image for use by the operator for initially locating the patient's retina, based on an image from the optical system in real time. A coaxial “scene” or visual target may be included in the visual field of the device so that a patient can fixate his or her eye on this scene and reduce eye motion. In addition to reducing eye motion, the location of this visual target can bring the fovea centralis into the approximate center of the CCD detector 22. In. devices intended for children, the scene may include a visually pleasant object such as a familiar animal. The fixating light may also exist as a separate optical system for use with the other eye.

In the currently commercially available Nidek NM100 Hand-Held Non-Mydriatic Fundus Camera, the liquid crystal display (LCD) (or other display) screen is typically located on a desktop power source that is attached to the hand-held camera by a cable. While such displays may be used in the exemplary embodiments, the LCD screen (or other display device) may be placed on the back of the hand-held camera unit, so that the operator can more easily locate the retina, having the patient's eye and the LCD screen in the same line-of-sight. The illumination system 12 and detection system 22 may include the Nidek NM100 Hand-Held Non-Mydriatic Fundus Camera, the Topcon TRC-50EX (TRC-NW5S/TRC-NW5SF) and Topcon TRC NW6S Non-Mydriatic Retinal Cameras, including one or two Pulnix TM-7EX CCD digital cameras to capture images at one or two wavelengths.

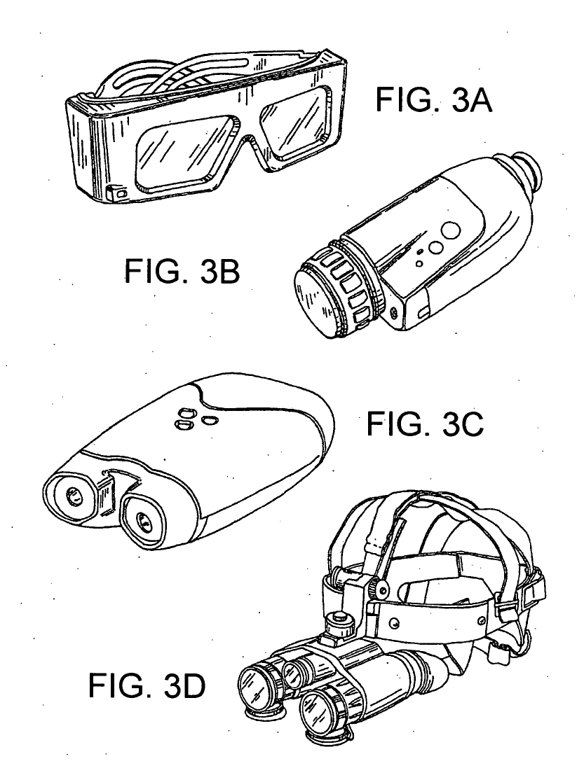

As mentioned above, the device may be operated by the patient as a self-testing device. The patient may place his or her eye near the lens of the device, aligning the eye with a pre-determined spot of light or a small scene. This device may be similar in size and form to currently-marketed virtual reality or night-vision goggles, as shown in FIG. 3a. Although exemplary embodiments may be used with a dilated eye pupil, the imaging of the retina may be carried out without dilation of the pupil for speed of measurement and patient convenience. The camera may include a shield (not shown) to prevent ambient light from entering the optical system 11 to minimize extraneous reflections and the introduction of optical noise.

Referring again to FIG. 2., the optical system 11 also interfaces with a locate and focus system 16, which utilizes feedback from an image capture system 17 (also interfaced to the optic system 11) to automatically find and bring the retina into focus. A convolver or other pattern recognition software may be utilized to locate the foveal region of the retina. After using the pattern recognition information to more precisely locate the foveal region in the center of the viewing field, the image may then be magnified using a series of lenses in the optics system 11 such that the foveal region fills a large portion of the active area of the CCD (or other detector). In some embodiments, the optical system tracks movement of the retina such that the foveal region is centered and occupies most of the optical field of view. The optical system 11 may be configured to track the motion of the retina through a motor drive system that slightly gimbals the lens system. This motion system is driven and controlled in a closed loop manner utilizing the feedback of the pattern recognition software. Alternatively, if the patient is able to keep his or her eye still during the measurement, the registration of images would not be required. In other embodiments, the location of the fovea may be tracked using software techniques to locate a portion of the image corresponding to the foveal region. The fovea is generally the darkest portion of the retina, and a computer or microprocessor can be programmed to locate the fovea using any combination of relative darkness, contrast, or a specific shape. This location can be determined for each frame of a series of video frames, and the reflectance measurement made in the corresponding foveal region of the retina in each frame.

To adjust for variations in the individual patient's refraction, a refractive adjustment such as a variable corrective lens with a thumbwheel adjuster may be incorporated into the device. Should changes in the patient's focus change during the measurement (e.g., during naturally-occurring accommodation), the image processing or optics can be adapted to compensate. This can be done by comparing the focus of successive images, and correcting the optical system using an electromechanical servo system to adjust focal position of the optics, or by known image-processing techniques in the computing system.

In some embodiments, the image capture system 17 is selectively controlled by the software (or alternatively by the operator) and uses feature and pattern recognition to drive the locate and auto focus system 16 to capture and store an appropriate image for analysis. Image capture itself is analogous to the function provided by a digital still camera. The initial image capture may be carried out with commercially available data capture boards such as a National Instruments N11409 installed in a computer such as a commercial PC. The image capture system 17 may utilize feature and pattern recognition to drive the locate and focus system to capture and store an appropriate image for analysis. Commercially available pattern recognition software including the mathematical tools in MATLAB may be used. An image analysis system 18 is interfaced with the image capture system 17 to analyze the light reflected from the retina to quantitatively determine the amount of glucose present. The results may be displayed to the operator via the output system 20. The output system 20 presents results together with any feedback associated with the acquisition of the data, and may include an LCD display screen or other display devices.

FIG. 3a illustrates one form factor of an analysis apparatus in conjunction with the eye of the patient, shown illustratively at 10 in FIG. 2. The analysis apparatus includes an optics system 11 comprised of lenses for projecting illuminating light onto the retina, directly through the pupil, and for receiving the light reflected from the retina passed out through the pupil, and for focusing that light to create a signal or to form an image. The glasses preferably include lensing to provide an optimal view of the retina to be illuminated and imaged. In such a system, glucose concentration information may be displayed to the user directly while the glasses are worn. When used in this form factor, in order for the device to be used conveniently by a patient, it is especially desirable that the weight and volume of the device be minimized, preferably to a weight of about ten ounces or less, and to a total volume of about twenty cubic inches or less.

FIG. 3b illustrates another form factor of an analysis apparatus in conjunction with the eye of the patient, shown illustratively at 10 in FIG. 2. The analysis apparatus includes an optics system 11 comprised of lenses for projecting illuminating light onto the retina, directly through the pupil, and for receiving the light reflected from the retina passed out through the pupil, and for focusing that light to create a signal or to form an image. The monocular device preferably includes lensing to provide an optimal view of the retina to be illuminated and imaged. In such a system, glucose concentration information may be displayed to the user directly while the monocular device is in use.

FIG. 3c illustrates another form factor of an analysis apparatus in conjunction with the eye of the patient, shown illustratively at 10 in FIG. 2. The analysis apparatus includes an optics system 11 comprised of lenses for projecting illuminating light onto the retina, directly through the pupil, and for receiving the light reflected from the retina passed out through the pupil, and for focusing that light to create a signal or to form an image. The binocular device preferably includes lensing to provide an optimal view of the retina to be illuminated and imaged. In such a system, glucose concentration information may be displayed to the user directly while the binocular device is in use.

FIG. 3d illustrates another form factor of an analysis apparatus in conjunction with the eye of the patient, shown illustratively at 10 in FIG. 2. The analysis apparatus includes an optics system 11 comprised of lenses for projecting illuminating light onto the retina, directly through the pupil, and for receiving the light reflected from the retina passed out through the pupil, and for focusing that light to create a signal or to form an image. The head-mounted device preferably includes lensing to provide an optimal view of the retina to be illuminated and imaged. In such a system, glucose concentration information may be displayed to the user directly while the head-mounted device is in use.

Following bleaching of the visual pigment with light at selected wavelengths, one embodiment uses the measurement of reflected light from the area of interest, which preferably is the foveal region of the retina (although any area of the retina that contains visual pigment could be used) to measure visual pigment regeneration. The retina, at specific wavelengths of light, is illuminated as described above, and the reflected light is captured by a sensing device as described above. This sensing device may be a CCD, a CMOS imager, a photodiode or any other device that can sense the amount of light being emitted from the eye in order to measure the regeneration of the visual pigment during or following bleaching. In one embodiment using imaging, the light values of the pixels (in the case of a CCD or CMOS imager) that are in a defined area containing the desired visual pigment to be measured can then be summed. Although the exemplary embodiments can be used to measure the changing light reflected off any defined area in the retina of the eye, it is preferred to measure the foveal area which contains the highest percentage of cones compared to rods. Although both cones and rods contain visual pigment, the regeneration of cone pigment is considered to be faster than rod visual pigment regeneration and therefore preferable for measurement of regeneration rates. The highest concentration of cone visual pigment is contained in the foveal region, which is the area of central vision.

Since several exemplary embodiments of this invention measure regeneration of visual pigment, the reflected light must be measured over a period of time, either with constant light or via a series of pulses. One embodiment makes the measurement of visual pigment regeneration with a series of pulses. This temporal measurement can be accomplished by comparing the reflected illumination from pulse to pulse, over a series of pulses, of the same area of the retina. A better estimate of the changing reflectance may be made by averaging the change in reflectance over a number of pulses to minimize noise. Although a large number of pulses may be used for greatest accuracy, it is generally desirable to use as few pulses as possible for patient convenience and comfort. A pulse is defined as any illumination of the retina, which may be a temporal illumination with any intensity, modulation and frequency. In addition, the illumination may be a steady-state illumination.

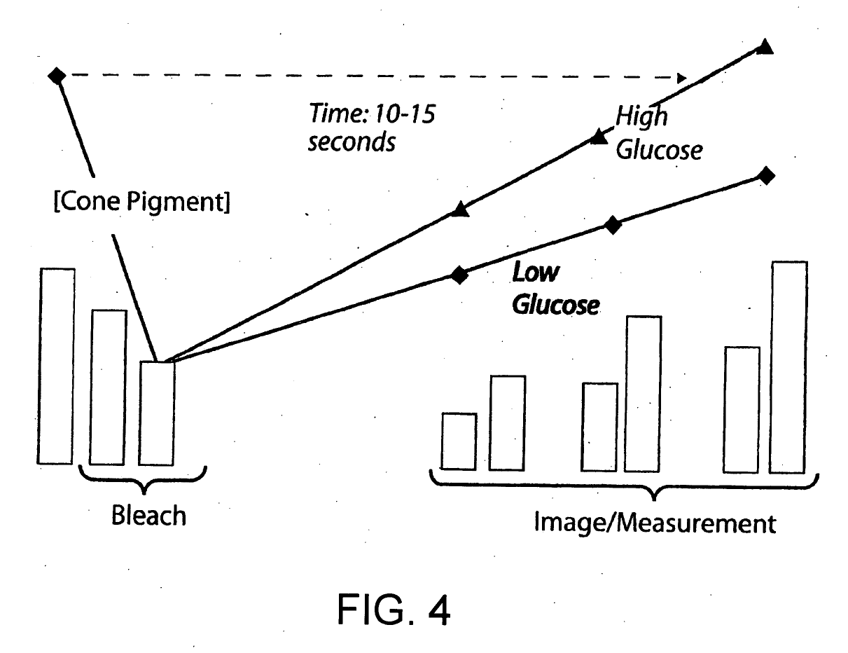

Various pulse sequences may be utilized comprising, for example, a pulse or series of pulses at wavelengths of light that cause the breakdown (bleaching) of the visual pigment, and then a series of pulses (possibly with less intensity than the pulses that were used to cause the visual pigment breakdown) used to illuminate the retinal area of interest, allowing for the measurement of the change in reflection of the area of interest and, thus, the content of the visual pigment. The wavelength of the illuminating light could be the same as the initial bleaching light or the illuminating light could be of different wavelength than the bleaching light. One exemplary pulse sequence comprises one to four strong pulses, to heavily bleach the visual pigment, and then a series of low intensity pulses applied over a selected period of time to allow images to be made. The change in reflected light is measured via these images, and the change versus time indicates the rate of regeneration, as illustrated in FIG. 4. By measuring the slope of the regeneration, the glucose concentration can be calculated. The higher the slope of the regeneration of the visual pigment, the higher the concentration of glucose. This curve is not necessarily linear, and the actual measured reflectance of the retina decreases as regeneration proceeds.

The wavelength of light chosen for the illumination pulses may be any wavelength that would be absorbed by any visual pigment. In a preferred method, narrow band light that is absorbed by either green visual pigment or red visual pigment may be used. It is preferable to avoid illumination light in the blue range, since blue light is more highly scattered by cataracts than the longer visual wavelengths; cataracts are a common malady in diabetic patients. The device may either use polychromatic light (e.g., the white light that is contained in currently marketed retinal cameras) for the pulse sequence, with the light then being filtered at the CCD or narrow-band light specifically chosen for a particular visual pigment (e.g., 540 nm light for bleaching of the green visual cone pigment, or a wavelength such as approximately 620 nm which allows higher intrinsic intensity and therefore better instrumental performance) for use as the illumination light. Narrow band light has two advantages. First, narrow band light is generally more comfortable for the patient and, secondly, the pupil does not react with as much constriction to each pulse of narrow band light as compared to broad-band light.

A background blue light may be used throughout the testing period to reduce the effect of the rod visual pigment, by keeping these pigments in a constant bleached state. Since the regeneration rate of this rod pigment is thought to be slower than cone visual pigment, the addition of pigments of differing regeneration times may lessen the accuracy of the measurement without this feature.

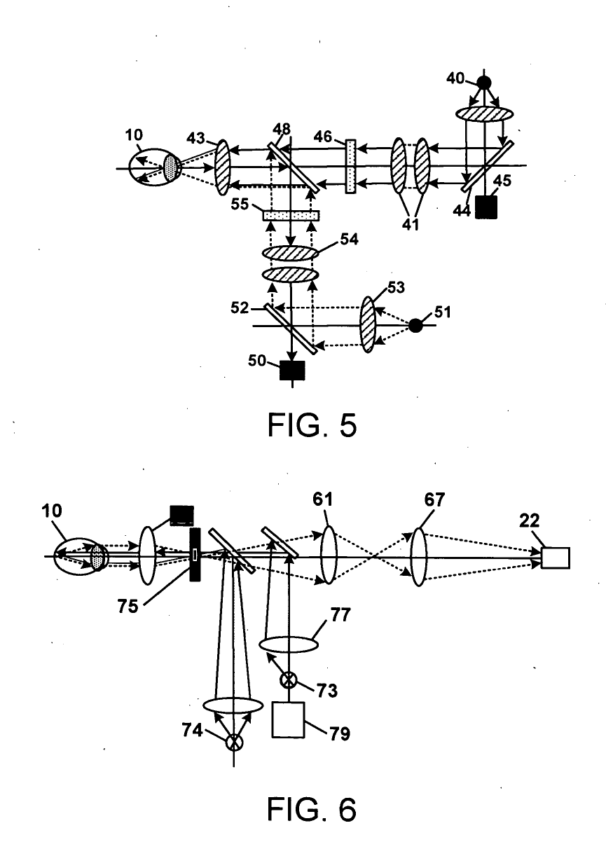

A further embodiment of the optics system 11 and illumination system 12 is shown in FIG. 5. This configuration provides a light source at one wavelength and a sensor system that operates with its own separate light source at a second wavelength. The use of two wavelengths completely separates and isolates the bleach light source from the sensitive measurement process. Thereby, a sensor that does not respond to the bleaching wavelength does not sense the bleaching light and its output can be amplified for the reflected light at a second wavelength.

In the horizontal path with the eye 10, a pulsed light source 40 is imaged into the pupil of the eye with sensor/source optics 41 and an eye lens 43. A sensor 45, near the pulsed source, is used only for feedback control of the pulsed source and receives light through a beam splitter 44. The pulsed source 40 is filtered by an interference filter 46 at 550 nm. The filtered light passes through a dichroic beam splitter 48, and then travels through the eye optics 43 and into the eye 10. This source and path accomplishes bleaching of the visual pigments with high intensity light. The bleached area is then monitored over time by sensor 50 coupled with lower intensity light at the second wavelength. The rate of recovery or rate of regeneration of the visual pigment is the parameter that is used to calculate the glucose level.

With reference to FIG. 5, the light path for measurement of the visual pigment regeneration (light going through elements 54 and 55) is provided to sense the very low reflected light levels without interference of the bleaching light, which may be of a different wavelength. This can be accomplished by operating a steady light source 51, with source optics 53, to illuminate the back of the eye at a significantly different wavelength to allow for total blocking of the 550 nm pulsed source. The source 51 light is combined with the sensor path with a beam splitter 52 passing through optics 54, and then is filtered to a narrow range preferably around 600 nm by interference filter 55. The source 51 light is focused at the pupil of the eye to provide light to a broad area of the retina. The sensor path may operate at 600 nm with the use of a filter 55, or at a wavelength significantly different than the wavelength of the pulsed source. A wavelength near 600 nm is a preferred choice because the long wavelength pigments in the cones are still very sensitive at 600 nm and the blood vessels in the retina absorb relatively little light. The steady light from the source 51 is at a low level that does little bleaching. The sensor 50 is conjugate with the retina of the eye and is thereby in focus with the retina. The sensor 50 can be, for example, a CCD, CMOS imager, or a photodiode. The photodiode can be a more sensitive device than a standard CCD and it can be utilized in the frequency domain to filter out all of the first order effects and only look at the higher order harmonics as described in the above-referenced U.S. Pat. No. 6,650,915, or to make other time-based, frequency-based, or phase-based measurements.

With reference to FIG. 6, another embodiment of the invention uses a pinhole 75 located confocally with respect to the retinal image. Light is projected into the eye through this pinhole aperture and reflected light from the retina is collected back through it. The confocal pinhole 75 serves to limit the spatial extent of the light on the retina. The size of the pinhole 75 may be changed to suit the requirements. For instance, it may be beneficial to illuminate only the foveal spot on the retina. By avoiding the illumination outside the foveal region, bleaching of rods would be minimized. Since cones regenerate faster than rods, this would expedite the measurement process.

Alternatively, it might be preferable in some subjects to make the measurement outside the foveal region. This could be especially true in subjects with macular degeneration. In this case, the confocal pinhole 75 could be annular in shape, allowing measurement of a spatial ring outside the foveal region. Also, the confocal pinhole 75 could contain a multiplicity of segments or holes. This would allow different portions of the retina to be illuminated by different types or levels of light. For instance, two spots of light could be projected onto the retina. The retinal reflectance would change in response to this light, and achieve a steady state after a period of time. Either during this equilibration process, or upon achieving steady state, the reflectance from these two or more spots is measured. The reflectance values and the difference between them are correlative with the level of blood glucose and can be used to measure the blood glucose level. The multiplicity of spots can be projected onto the retina in any arbitrary pattern, possibly as an array of spots in a grid, or as segments of a circular spot. The light spots can be detected either with discrete detectors or with a single array detector such as a CCD array. The measurement method described here can give a very rapid measurement of blood glucose. As equilibration is reached over a short period of time, the noise in the measurement decreases. In addition, this measurement, made in a light adaptation (bleaching) phase, can be made at relatively high light levels compared to measurements made purely in the regeneration, or dark adaptation, phase.

In an embodiment with CCD or CMOS imaging, image analysis tools available in commercially available software packages such as MATLAB can be used. With these tools, the image overlay can be accomplished so that the exact area is repeatedly measured. The initial image capture can be accomplished with a commercially available data capture board (e.g., a National Instruments NI 1409 installed in a PC) and the mathematical tools in MATLAB can then be used to analyze the trends in the regeneration rates and to convert those values to glucose levels.

In one variation of the photodiode measurement of the reflectance, a CCD or similar device is used to “steer” the photodiode to the area of interest (e.g., the foveal region). The photodiode integrates the signal from an area whereas the CCD provides an image. With a sufficiently sensitive CCD, formation of an image allows the definition of an area to be measured, and that area can be repeatedly measured. If a photodiode is used, it may need to be aligned to the spot to be measured, which can be done with known servo methods.

A consideration in making comparable measurements is the variation in light that illuminates the area of interest due to the pupil changing size and to head/eye movement during the capture of the repeated images. This variation can be minimized by also making measurement of a non-changing target in the back of the eye. The optic disk is a good choice of an area to measure and may be used as a reference. For example, this may be done by calculating a ratio of the light returned from the measurement area to the light returned from a defined area of the optic disk. The optic disc is area of the retina where the optic nerve enters the eye. It contains nerve fibers but no cones or rods. Another way to establish a reference is to take measurements at two wavelengths of light, with one wavelength selected for strong absorption by a cone visual pigment, e.g., green at 540 nm, and the second at a non-absorbing point, e.g., 800 nm. The area of the retina to be used for image stabilization can be illuminated by light of a wavelength outside the wavelengths absorbed by visual pigment, and spatially or spectrally distinct from the area used to measure regeneration. For instance, near infrared wavelengths longer than 700 nm can provide excellent contrast of retinal vasculature. An annular ring image using such near infrared wavelengths could be used.

In embodiments that use imaging, bleaching can be done over a greater area than that which is to be measured. By establishing datum points from a first image following bleaching, and then measuring the darkness of a defined area relative to the datum points, subsequent measurements can again measure the same area by reference to the datum points. Alternatively, the first image can be used as a filter which is passed over the subsequent data, and by known image processing methods of translation, rotation, and scaling, the exact overlay can be obtained to thereby locate the same area. The measure of brightness of the defined area is accomplished by summing the value of all of the pixels of the camera in the defined area.

FIG. 6 illustrates an exemplary apparatus to quantitatively measure light reflected from the human retina. The device uses an imaging CCD camera 22, onto which an image of the retina is placed. A region of interest can be selected based on the experimental requirement. For example, the device can image a spot of the retina that is physically 0.6 mm in diameter. A larger spot can be imaged using a larger pinhole aperture. Although FIG. 6 shows a second LED 74 that could be used for measuring regeneration at a second wavelength, in the examples that follow, a single LED 73 with a wavelength of 593 nm was used as illumination for both the bleaching phase and for the regeneration phase.

The head is brought into position and rested in a head restraint consisting of an adjustable chin rest and forehead strap. The head restraint is adjusted to bring the eye to a position where it is possible to look into an eyepiece 63. The eyepiece 63 can be a standard 10×wide field microscope eyepiece, such as the Edmund #A54-426. The retina is illuminated with light from a 593 nm wavelength LED 73, such as a LumiLEDS #LXHLMLIC LED with adjustable intensity controlled from a DC power supply (e.g., CIC PS-1930). The output of the LED 73 can be measured with a power meter 79, such as the Melles Griot 13PDC001. The LED emission is collected with a 10×microscope objective lens 77, such as Edmund #36-132. The LED 73 is re-imaged onto the reticle plane of the eyepiece 63. For example, a 1 mm pinhole aperture 75 is located at this reticle plane and serves as a confocal aperture. The area of the illumination is limited by this aperture to 1 mm. The magnification power of the eyepiece 63 and of the human eye combine to make the final image diameter on the retina equal to 0.6 mm diameter in this example. The power meter 79 is used to adjust the power density at the retina from LED 73 to the level required for either the bleaching or regeneration phase-in this example 5.8 or 4.2 log Trolands, respectively. (Troland is a unit of measure of retinal illuminance defined as 1 candle/m² on a surface viewed through an artificial pupil of area A=1 mm²)

The subject is directed to look forward into the eyepiece 63, so that the image of the pinhole is centered in his field of view. As a result, the light is imaged onto the foveal spot of the retina. A portion of the illuminating light is reflected by the retina and passes out through the pupil of the eye, through the eyepiece 63 and is imaged confocally onto the 1 mm pinhole. The light passed by the pinhole then impinges on two 4×microscope objective lenses 61, such as Edmund #36-131 lenses acting as a relay lens system. The image is carried along further and eventually the retina and pinhole are imaged onto the active element of the CCD camera 22, such as a Pulnix #TM-1020CL or DVC #1412AM camera.

The digital images are collected from the camera 22 using a CameraLink T frame grabber, such as National Instruments #1428 installed in a PC. The files are saved as discrete images and formed into a multi-layer file. An exemplary analysis procedure is as follows. The camera 22 is set to the highest gain setting and binning is set to 2×2. A series of raw images is collected. Initially the LED is at low intensity. After 2-3 seconds the LED is switched to high intensity and left high for 20 seconds for the bleaching phase, then switched low again. The regeneration is measured for about 40 seconds at the low light intensity. The data collection results as a series of image files. A 40×40 pixel region of interest (ROI) is defined, in the center of the bleached foveal region. The mean intensity within the ROI is found for each image, and the mean intensity data are exported to a spreadsheet program for display and analysis.

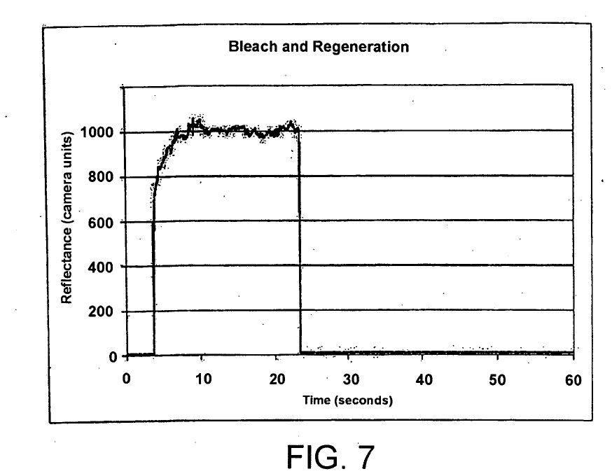

FIG. 7 shows a graph of an example trace. Each data point is the mean intensity within a region of interest in a camera frame. The camera frame rate is 20 frames per second. The x-axis shows time in seconds. The y-axis shows mean pixel intensity in camera units. In FIG. 7, it can be seen that when the LED is switched to the bright setting at about the 3 second point, the measured signal first increases rapidly, but then a slower increase in retinal reflectance (due to bleaching) can be observed. When the LED is switched low at 23 seconds, the regeneration of visual pigment can be followed. Intensity points immediately before and immediately after the light is switched from high to low intensity can be used to photometrically correct the measurement system, since the ratio of the input light intensities is known with a high degree of accuracy. The ratio of the reflected and measured light intensities should have the same ratio, assuming that the measurement circuitry is linear. If the ratio is not the same, it can be due to the introduction of an offset on the intensity axis. An algorithm can be used to remove any offset, thereby creating an intensity axis in true spectroscopic units of percent reflectance, as a percentage of the full bleach. This technique could be considered to achieve the same result as having measured a background trace at full bleach, but it arrives at a photometrically accurate result without degrading the signal-to-noise ratio of the data from division by a second noisy signal.

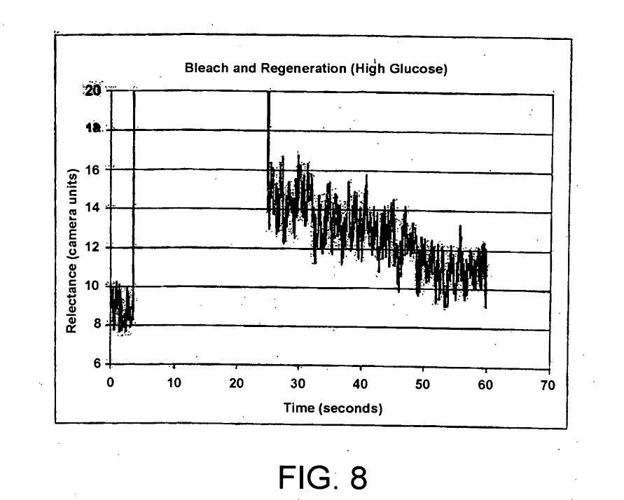

FIG. 8 illustrates an expanded view of a portion of the graph of FIG. 7, showing the lower level reflectance values in greater detail. In the above experiment, the glucose level of the subject was 123 mg/dl. At the start of the experiment, the reflectance of the foveal region is relatively low, measuring about 9 camera counts. The subject had been in a normally lit room prior to the experiment. The reflectance level can be considered indicative of the reflectance level of the retina for this subject in normal room light. At the 3 second point, the LED is turned high and the retina begins to be bleached, thus becoming more reflective. When the LED intensity is returned to the original level, it can be seen that the reflectance of the retina is higher than it was before, now measuring about 15 counts. Over time, the reflectance decreases, following a fairly linear slope until 55 seconds, where it proceeds at a slower rate of regeneration.

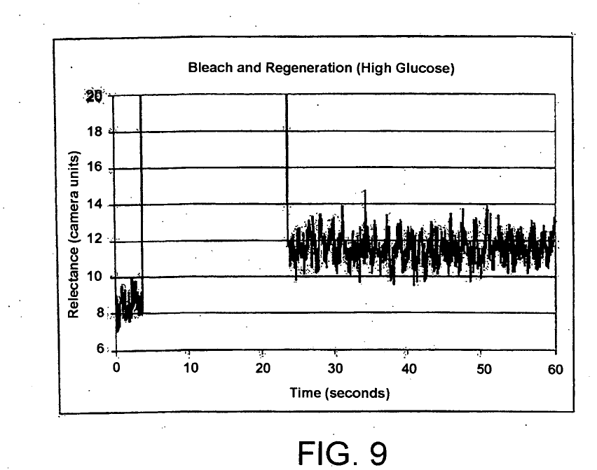

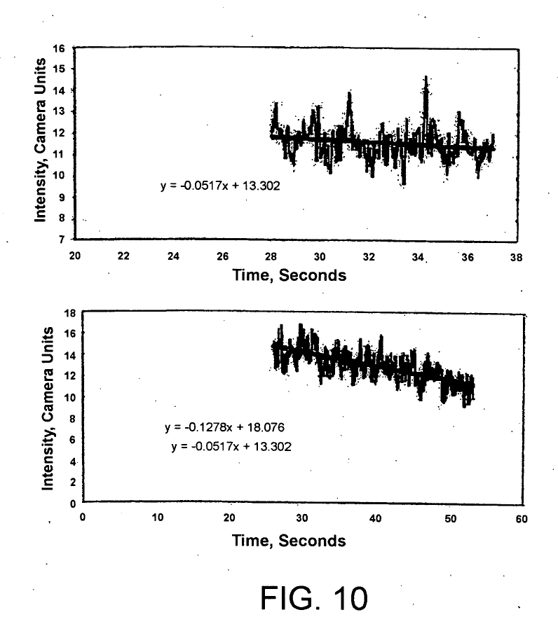

FIG. 9 shows a graph depicting measurement from the same subject, when his glucose level is low, at 81 mg/dl. In this measurement, reflectance again starts out low, at 8-9 camera counts. Following the bleach event, the reflectance is about 11-12 camera counts. Instead of rapidly decreasing, the reflectance remains near this level over the course of the remaining roughly 40 seconds. The initial downward slope of the regeneration curve following bleach is the quantity that is used to correlate with glucose level. A linear portion of the regeneration data near the beginning of the post-bleach phase is extracted and a best-fit line is calculated. Linear fits for regeneration curves are shown in FIG. 10, where the top graph is a low glucose reading (81 mg/dl) and the lower graph is a higher glucose reading (123 mg/dl).

Other methods of measuring blood glucose also rely on measuring a regeneration rate of a retinal pigment. For example, pulsed light techniques such as those described in U.S. Pat. No. 2005/0,010,091 may be used to illuminate the eye. According to another exemplary embodiment, glucose is measured using the rate of bleaching. Since regeneration is occurring whenever the eye is not completely dark-adapted, faster regeneration reactions which occur at high glucose concentrations would slow the rate of bleaching. This relationship provides a methodology of measuring regeneration rate, and thus glucose. First, the light is brighter and, therefore, easier to see with an inexpensive camera. Second, the reaction goes faster, making the test possibly shorter in duration. Third, there is no need for “registration” of frames between a bleach phase and a regeneration phase. Lastly, regeneration can be measured without causing additional bleaching from the measurement pulses.

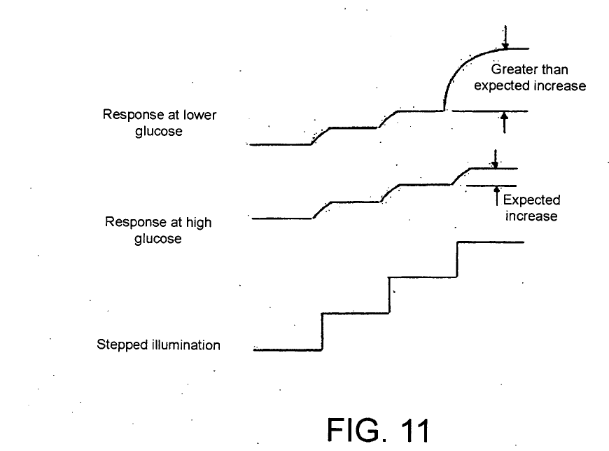

In yet another embodiment, illustrated by FIG. 11, blood glucose can be measured using the regeneration of visual pigments without a “bleaching event.” In one example, referred here to as steady-state regeneration measurement methodology, glucose is measured by determining retinal reflectance at different light levels. This is the equivalent of the color matching methodology described in U.S. Pat. No. 2004/0,087,843. At a given light level, if the glucose concentration is high enough to regenerate the pigment at a rate higher than that bleached by the light, a fixed level of reflectance (calibrated for each patient) results. When the light level causes more bleaching than can be regenerated, the visual pigment is depleted faster than it can be made, and the reflectance level rises to a level higher than if a higher concentration of glucose was present. In this method, the retina is illuminated with one light level, a steady state is achieved, and the reflectance is recorded. The retina can be illuminated at a second, increased level, and a new steady state reached. This reflectance is recorded and calculated as a ratio to the first reading. If the light level is still below that which causes more bleaching than regeneration, the expected increase in reflectance results. If, however, the new light level causes more bleaching than regeneration, a higher reflectance than expected would be measured at the new light level. If the light levels are increased in a step-wise fashion, eventually a level is reached where the bleaching effect of the light exceeds the regeneration rate for the patient's glucose level, and a higher than expected increment of reflectance results (a “threshold effect”). Estimation of glucose can be made by considering the light levels below and above the threshold, and from the change in the ratio from the expected amount.

In a second example of measuring blood glucose using visual pigments without a “bleaching event,” a steady-state regeneration measurement methodology uses measurement pulses only to create a steady state of foveal reflectance which corresponded to glucose level. The first pulse increases the reflectance of the foveal region, and each pulse is adjusted to maintain the same reflectance. This procedure is repeated at a second illumination level. The levels of reflectance measured during the initial pulse and the second pulse, as well as the ratio of the magnitude of the pulses required to maintain the same reflectance reading at the two levels, are related to glucose concentration.

When glucose measurements are sought, there may be patient-to-patient variability, and the calibration of each device may be required owing to this variability. Also, as the changing state of each patient's diabetes can affect retinal metabolism and thus influence regeneration rates of the visual pigment, re-calibration may be required at periodic inteverals. Periodic calibration of the device is useful in patient care as it facilitates the diabetic patient returning to the health-care provider for follow-up of their disease.

Owing to the simple optical systems employed in the foregoing embodiments, and the absence of any requirement to separate the different wavelengths of light for spectral analysis, it is practical to make these embodiments from readily-available, lightweight, small optical parts (e.g., a CCD and lenses), and to construct the devices in the form of glasses, goggles sufficiently small and light to be comfortably worn by the user, or in the form of small hand-held devices such as monoculars or binoculars. Similarly, a small head-mounted device with a weight low enough to be comfortably worn by the user can be constructed from these components.

Examples of Clinically-Acceptable Glucose Measurements

Table 1 shows the slope (regeneration rate) obtained for 16 regeneration experiments on 6 different days, using three different subjects, with the apparatus depicted in FIG. 6. For these measurements, a single LED with a wavelength of 593 nm and two brightness levels was used for both the initial (bleaching) illuminating phase, at high brightness, and for measurements of reflective during the subsequent regeneration phase, at low brightness. The bleaching was carried out over a 20-second period, and the slope of each regeneration was subsequently recorded using the CCD array over a period of time, as described above in the detailed description of FIGS. 6 through 10.

TABLE 1

| | | | Slope | .abs slope | Calculated | Reference |
|---|---|---|---|---|---|---|
| Subject | date | trial# | (cts/sec) | (cts/min) | Glucose | Glucose |
| RGM | 2-Apr | 1 | −0.1233 | 7.3980 | 129 | 148 |
| | | 2 | −0.0877 | 5.2620 | 113 | 106 |
| | | 3 | −0.0386 | 2.3160 | 89 | 93 |
| | 3-Apr | 1 | −0.1058 | 6.3480 | 121 | 132 |
| | | 2 | −0.0390 | 2.3400 | 90 | 100 |
| | 4-Apr | 1 | −0.0857 | 5.1420 | 112 | 118 |
| | | 2 | −0.0309 | 1.8540 | 86 | 101 |
| | | 3 | −0.0353 | 2.1180 | 88 | 89 |
| RHS | 6-Apr | 1 | −0.0693 | 4.1580 | 104 | 96 |
| | | 2 | −0.331 | 19.8600 | 228 | 163 |
| | | 3 | −0.0391 | 2.3460 | 90 | 109 |
| JW | 8-Apr | 1 | −0.1976 | 11.8560 | 165 | 191 |
| | | 3 | −0.273 | 16.3800 | 200 | 202 |
| RGM | 12-Apr | 2 | −0.0517 | 3.1020 | 96 | 81 |
| | | 3 | −0.0930 | 5.5800 | 115 | 104 |
| | | 4 | −0.1279 | 7.6740 | 132 | 123 |

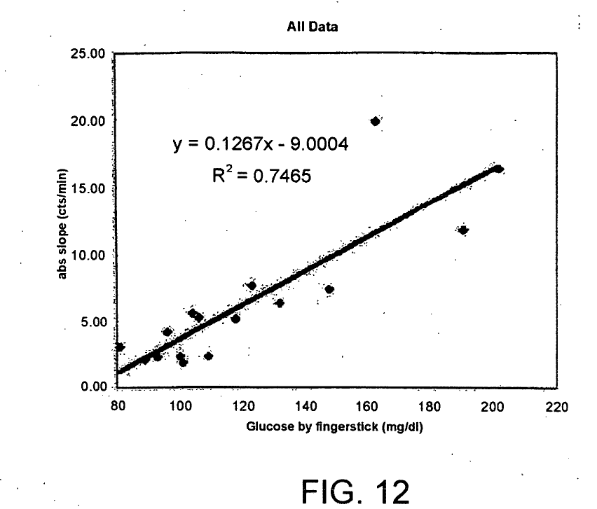

These slopes (or rates) are plotted against the reference glucose concentration, and a best-fit line is computed. These results are shown in a graph depicted in FIG. 12.

The linear fit line is now used to compute a glucose value (x) for a given slope (y). Each of the sixteen experiments is analyzed in this manner, resulting in the “Calculated Glucose” column of Table I which may be compared to the “Reference Glucose” column to the right, which are values obtained for the subjects with a conventional blood glucose meter.

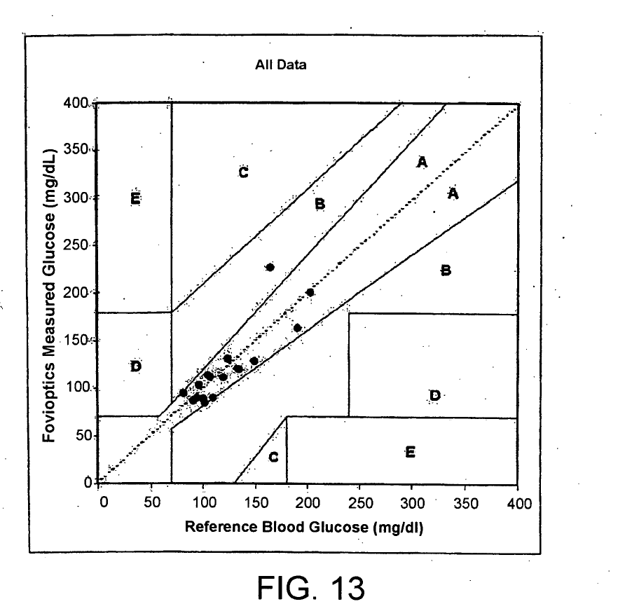

All of these data are plotted on a Clarke Error Grid, shown in FIG. 13. In this graphical grid system, which is used to evaluate the clinical impact of errors in blood glucose measurement, fifteen of the sixteen data points fall in region A, and one data point falls in region B. The regions of the Clarke Error Grid are defined as: A: “Clinically Accurate,” B. “Benign Errors, Clinically Acceptable,” C. “OverCorrection,” D. “Dangerous Failure to Detect and Treat,” and E. “Erroneous Treatment, Serious Error.” These results therefore constitute clinically-acceptable accuracy for the measurement of blood glucose using this technique.

In addition, the data shown in FIG. 13 were collected over the eleven-day period from April 2 through April 12. All the data are plotted on the graph based solely on the reflectance change measured during a period of time, with no intervening calibration or recalibration of the relationship between the rate of regeneration and the corresponding glucose value. Thus, it can be seen that at least over an eleven-day period, there was no need to adjust the response of the measurement due to environmental or physiological changes in the patient, and a recalibration interval for the device equal to or longer than eleven days can be inferred from the accuracy of the results obtained.

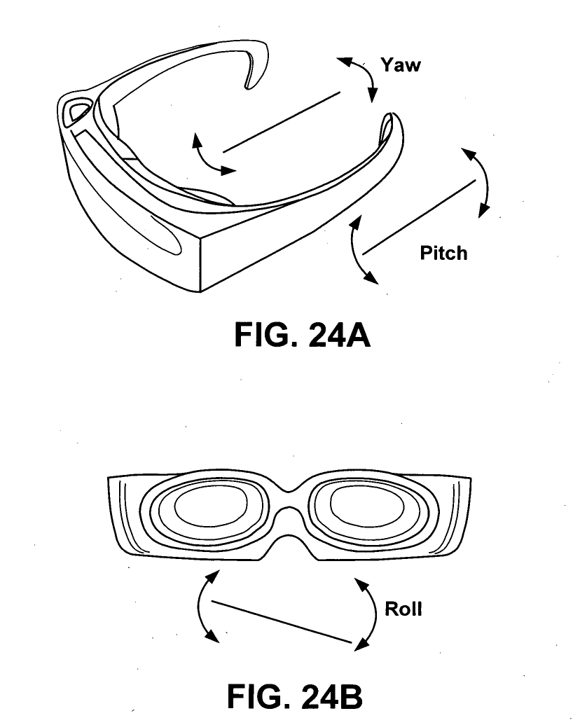

Under some approaches to determining blood glucose concentration, it is necessary to illuminate the eye with a beam of light and to detect the amount of light reflected from the retina over some period of time. To provide reliable illumination for quantitative measurements, it is necessary to maintain registration between the eye and the beam of illuminating light. There are two primary aspects of maintaining this registration, the first of which is establishing the direction of the patient's gaze (with gaze direction being defined as resulting from rotation of the eyeball about any combination of vertical and horizontal axes), which is generally described by the term “gaze fixation.” The second aspect relates to preventing or minimizing translation or rotation between the eye and the beam of light. Motion can consist of translation in any of three axes: X-axis (horizontal), Y-axis (vertical) or Z-axis (in-out movement toward or away from the eye), or in three axes of motion generally described as roll (rotation of the light beam about a center line between the light and the eye), pitch (tilting up or down of the light beam relative to the eye), or yaw (sideways movement in a horizontal plane), as shown in FIG. 24. All six motions are indicated here by “head movement,” which term includes movement of the head and eye with respect to the instrument, and vice versa.

A third aspect of this registration between the eye and the illuminating light beam is that of calibration and repeatability of positioning. When the device is initially provided to the patient, some adjustment and alignment will be necessary, either by the patient or by a technician, and may be done in a physician's office, clinic, or retail store. It is necessary to establish an initial alignment which will be used to create a calibration for the instrument; that is, the relationship between either level or rate of change of visual pigment and glucose concentration must be established for each patient. This initial calibration is stored in the device and used to calculate the glucose result for all subsequent measurements made with the device, or until the device is recalibrated at some future time.

Adjustments to achieve initial positioning of the instrument with respect to the subject's eye can be made in such a way as to best accommodate variations in the shape of the subject's head, the location of the eye with respect to other features of the head such as the nose and ears, clearance between the eye and the first optical element of the device, and other variations as occur among the population. Some adjustment may be performed as with eyeglasses, using elements such as the distance between temple pieces or the length and fit or the pieces that surround the ears. It is generally desirable that adjustments to the position of the optical beam be made while the amount of reflected light is monitored using either electronic measurements or a video system, and it is also desirable to allow the person fitting the device to do so without touching it and thereby changing its location. This may be accomplished by the use of motor-driven adjustment screws connected by flexible drives, operated by a computer or under the either the patient's or the fitter's control. Either person may make the necessary adjustments to bring the light beam into alignment with the eye of the patient.

A complicating aspect of the fitting and adjustment of such a device is that each person has a point of maximum forward reflectance of their retina (the “Stiles-Crawford effect,” Stiles, W. S. (1938) “Directional sensitivity of the retina and the spectral sensitivities of rods and cones”, Abstracts-Papers Communicated to The Royal Society of London, pp. 141-142; Stiles, W. S. (1938) “The Luminous Efficiency of Monochromatic Rays Entering the Eye Pupil at Different Points and a New Colour Effect”, The National Physical Laboratory, pp. 90-118.), and the location of this point varies among people (Applegate, R. A., Lakshminarayanan. V., “Parametric representation of Stiles-Crawford functions: normal variations of peak location and directionality,” J. Opt. Soc. Am. A., 10, Jul. 7, 1993, 1611-1623). It has been found that this point is frequently not located in the center of the pupil, and for maximum sensitivity and repeatability, the light beam should preferably be directed to that point. When the device is removed and then placed on the head again for a subsequent measurement, the beam should be returned to the same position to maintain the calibration for accurate measurements. The use of light beams, visible to the patient in the apparatus, of various size, shape, color, intensity and modulation, is helpful in establishing and maintaining this critical relationship of location and direction between the eye and the device.

The amount of light that can be shined into the eye is limited by safety standards (most recently published by Sliney D, Aron-Rosa D, DeLori F, Fankhauser F, Landry R, Mainster M, Marshall J, Rassow B, Stuck B, Trokel S, West T M, Wolffe M: International Commission on Non-Ionizing Radiation Protection, “Adjustments of guidelines for exposure of the eye to optical radiation from ocular instruments: statement from a task group of the International Commission on Non-Ionizing Radiation Protection (ICNIRP)”. Applied Optics 44 (11), 2162-76, April 2005). The amount of light returned from the retina to a detector for measurement is limited by two factors: the fraction (or the percentage) of light reflected from the retina (reflectance), and the portion of the reflected light which is directed toward and out through the pupil (the transparent opening in the iris) to the detector. At visible wavelengths, the reflectance of the retina is between about one and three percent, meaning that only a small amount of the light incident on it is reflected (the rest being absorbed or scattered). Furthermore, much of the retina acts as a diffuse reflector of light, causing the reflected light to be distributed over a relatively large angle, only a small portion of which passes through the pupil to a detector. Measurements of retinal reflectance show the amount of light reflected back through the pupil is generally on the order of 0.01% of the amount of light entering the pupil.

As a result, the amount of light available for measurement is very small (approximately four orders of magnitude less than the amount allowed to enter the eye in compliance with safety standards). A complicating factor is that some of the light incident on the eye is reflected from the front surface of the cornea (the “corneal reflection”), and is generally on the order of 1% of the light or approximately 100 times brighter than the retinal reflectance. Because the corneal reflectance comes from a point approximately 24 millimeters forward of the retinal reflectance (an adult eyeball is approximately 24 mm in diameter), in common optical systems the two reflections move different distances when the alignment between the eye and the light beam is altered. Thus, the relatively bright corneal reflection can complicate accurate measurement of the small amount of light reflected from the retina. In addition, since the amount of light falling on both the cornea and the retina can change with either position or angle of the illumination, the amount of light reflected can also change as a result of changes in the alignment or orientation of the eye and the light beam.

While minor changes in rotation of the eye may be compensated by retinal tracking or foveal tracking as discussed above, these approaches are more successful in correcting eye movements over a relatively small angular movement, such as of a few degrees, and the general direction of gaze still needs to be maintained in the general direction of a lighted target spot. Gaze-fixation techniques may be combined with the glucose monitoring approaches described above to maintain alignment between the foveal region of the retina and the instrument during the period of the measurement, which may be, e.g., 2 seconds or more. For example, if a visually attractive or stimulating target is provided, there is more of a tendency for the gaze to be directed toward the target and to remain fixated on it for a longer period of time. Approaches to making a target attractive can include using an shape or image with attractive content, and also by varying the color, intensity, rotation or shape over time.

In the case of the glucose measurements described above, there are additional possibilities and requirements for such a fixation target. As described above, in some approaches to measuring glucose using changes in visual pigment, the pigment is first depleted by a circular spot of bright light, then monitored with a less bright light as the pigment regenerates. When visual pigment is depleted, the ability of the visual system to respond to stimuli is reduced, and the target is no longer visually attractive to the user. This can be circumvented by the use of a color for the target which is different from the bleaching light color used. There are three varieties of cones, traditionally described as blue, green and red, but now formally labeled short, medium and long-wavelength cones. If the cones which detect red light are bleached by red light, the blue cones are still active, and a blue target, even in the center of a red image, is still quite visible and retains its attractive target properties. Other features of the target, such as blinking, fluctuations in brightness, movement or rotation will also serve to enhance the visual attractiveness of the target.

The second aspect of aligning the illuminating beam with the eye involves the six forms of translation or rotation collectively identified above as “head movement.” In addition to making the device as stable as possible in front of the eye of the user, it is sometimes necessary to provide information to the user regarding the relative stability of the eye with respect to the device. Approaches which can provide user feedback about position and absence of movement include monitoring the location and movement of a reflection from a portion the user, such as a reflection from the front surface of the cornea, detection of the relative location of the image of the retina within the instrument, and comparative location of various features on a sensor array in the instrument.

An effective means for indicating the presence of and reducing head movement, and for re-establishing the correct position after the device is removed and replaced for subsequent measurements, is to provide the user with a pair of shapes in the field of view, such as concentric circles of light of different color or intensity. One embodiment of this approach consists of a second color of light surrounding the image of the light beam used to illuminate the eye. This “surround light” is arranged so that the light beam overfills the pupil of the eye, and the user sees a slightly distorted circle which is the outline of his or her own iris where it blocks the beam. The device is initially aligned so that when the head is in the proper location, the surround light circle is concentric with the image of the illuminating light beam. When the proper optical arrangement of the images is provided, the position of the surround light image moves with any head movement while the image of the illuminating light beam remains steady, and any movement of the head causes the two circles to appear eccentric. The resulting eccentricity is easily detected by the user and provides feedback to return the head to the proper position, where visual concentricity of the two circles is restored. A procedure utilizing a “pupil shadow” similar to this has been described for marking of the cornea prior to surgery in Davis U.S. Pat. No. 5,013,319.

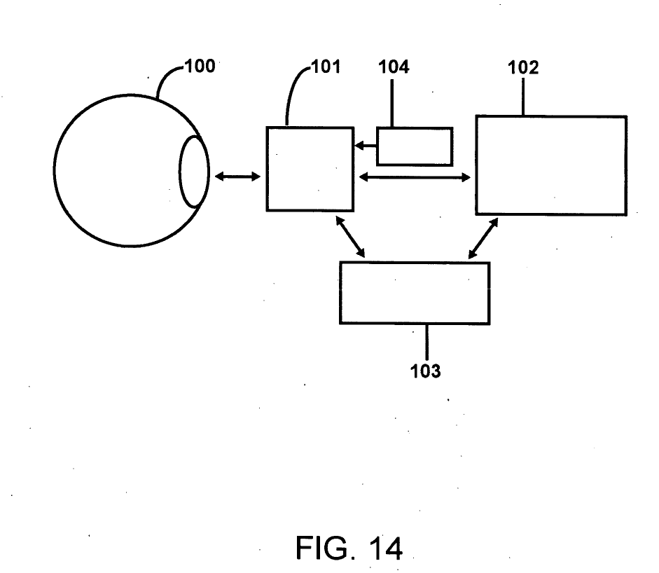

FIG. 14 illustrates a generic optical measurement system. An optical system 101 directs light from an illumination system 102 into the eye 100 and directs light reflected from eye 100 to a data capture and analysis system 103, which comprises elements used to perform a measurement or analysis. The fixation system 104 is an optical system for introducing a fixation object into the optical pathway of illumination system 102, optics system 101 and eye 100. The fixation system 104 assists the patient in maintaining eye 100 in a desired orientation during testing, data collection and/or analysis as performed by the measurement system 105. Fixation system 104 is illustrated between optics system 101 and illumination system 102 as an exemplary location. Those of ordinary skill will realize that the physical positions of the various system components may vary greatly depending upon the particular optics used and the specifics of the system. Fixation system 104 need not be physically between the other components but rather located in a position and displaying a fixation object perceptible to the eye and consistent with the other aspects of the present invention. Additionally, measurement system 105 may be a standalone system instead of a combined system (i.e., combined with illumination system 102).

Illumination system 102 provides selected illuminating light for illuminating as desired for performing a measurement in the eye. Illumination system 102 may, for example, use a monochromatic or multiple discrete wavelength light source that provides light for imaging the retina. In one embodiment, illumination system 102 provides light for imaging coaxially to reduce the likelihood of extraneous reflections from the interior or exterior of the eye. The light from illumination system 102 is projected through the pupil, using optics system 101. The wavelength of this light source is selected dependent upon the particular measurement, analysis, operation or procedure being performed by measurement system 105. Although any visual wavelength of light could be used, the light intended for absorption by visual cone pigments could be centered at 540 nm for green cones and 585 nm for red cones, or at other visible wavelengths selected for contrast, sensitivity or signal-to-noise ratio. Illumination light may be composed of two (or more) separate lighting systems, such as a xenon strobe, multiple laser diodes, or light-emitting diodes (LEDs).

In one alternative embodiment, fixation system 104 is used to display a fixation object to maintain eye gaze in a manner not to impede or interfere with the measurement, illumination or analysis being performed in the eye. In another alternative embodiment, the fixation object is selected and displayed using the fixation system 104 so as to remain within the visual field of a subject during a procedure. In another alternative embodiment, the fixation object is provided by the fixation system in such a manner that the fixation object remains perceptible to the subject during a procedure. In another alternative embodiment, the fixation object is provided by the fixation system in such a manner that assists the subject in remaining fixated on the fixation object during a procedure.

The utility of the fixation object and fixation system may be better appreciated by considering the operation of the eye. While the entire retina is sensitive to light and provides vision, the fovea is the only area of the retina possessing sufficient resolution and color discrimination for such tasks as reading, color comparison, or fine shape recognition. Subsequently, whenever the eye is required to perform such tasks, the fovea is directed to the particular area to be interpreted. However, the fovea occupies only a very small portion of the overall retina. The patient's gaze must remain fixated or directed towards the particular area needing interpretation until the task is accomplished. Thus, gaze fixation or fixing the gaze refers to the process of directing and maintaining the eye (and the fovea) into a desired position for testing and analysis.

In most cases, it is desirous that the fixation system does not impede or interfere with the functions or procedures performed in the eye by the optics system and the illumination system. In one embodiment, the fixation system uses a different wavelength of light than the illumination system light. In another embodiment, the fixation system uses the same wavelength of light as the illumination system light but is modulated or otherwise conditioned so as to minimize or eliminate any interference with the functions or procedures performed in the eye by the optics system and the illumination system. In still other embodiments, the fixation system uses multiple wavelengths of light to produce a fixation object.

In addition to advantageously selecting a wavelength or wavelengths for the fixation system, the fixation object itself may also assist the subject or patient undergoing the procedure or measurement in maintaining a gaze fixed in a desired position. In one embodiment, the fixation object may change or morph into different shapes, objects or images or move in and out of focus. For example, the fixation object may be the presentation of a series of letters. The subject's interest is maintained along with visual focus by attempting to identify, for example, a word or phrase spelled by the series of letters. The fixation object could be any of a number of objects or images presented in the visual field, such as, for example: shapes, letters, numbers, alphanumeric characters, or images (i.e., animation, photos or video).

In a still further advantageous embodiment, the fixation object may move. In one embodiment, the fixation object moves by rotating about a point within the visual field. In another embodiment, the fixation object moves by translating across or within the visual field. In another embodiment, the fixation object moves but remains constrained in movement so as to remain within and not disrupt the illumination, measurement, analysis or other procedure being performed by the illumination system and the optics system. In another embodiment, the fixation object moves but remains constrained in movement so as to remain within the capabilities or the field of view of the data capture and analysis system. For example, the movement of a fixation object is limited to maintain the aspect of the eye relative to an imaging system used. An imaging system useful in examination, imaging or measurement of the eye includes, for example, CCD, photodiode or other imaging system known to those of ordinary skill in the art.

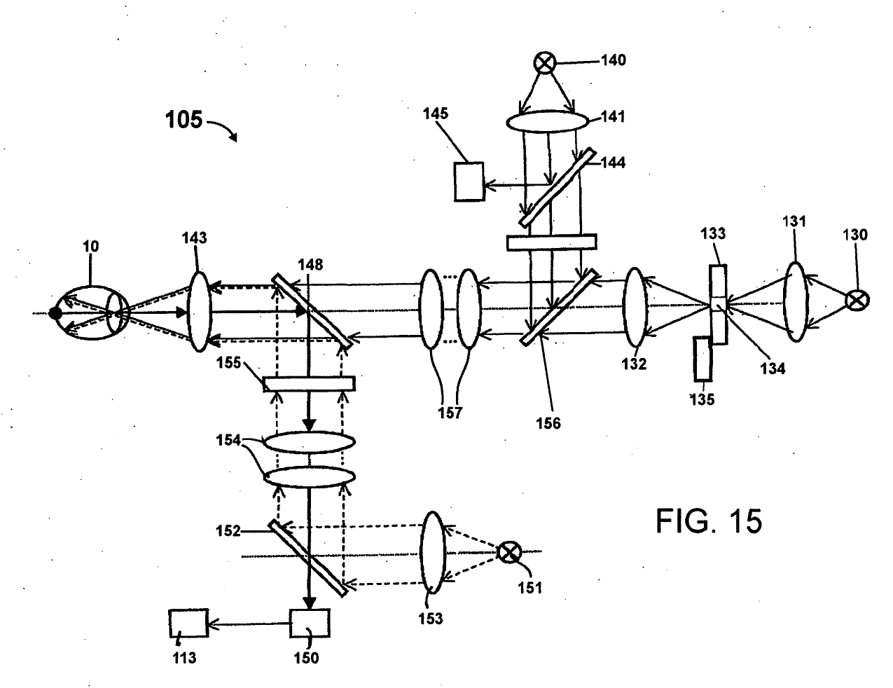

One embodiment of the arrangement of components in an integrated optical system 105 is illustrated in FIG. 15. Integrated optical system 105 includes an optics system, an illumination system, a fixation system, a measurement system, and a data capture and analysis system. In this embodiment, light sources and optical components are provided separately for each system. Those of ordinary skill will appreciate that these components may be arranged in a wide variety of configurations and that existing optical techniques would allow several components and/or light sources to be combined without distracting from the functional capability of the integrated optical system embodiments described herein.

In this configuration, measurement system operates a light source 151 at one wavelength, the illumination system operates with its own separate light source 140 at a second wavelength and the fixation system operates with its own separate light source 130 at a third wavelength. The use of multiple wavelengths completely separates and isolates the illumination light source from the sensitive measurement process performed by the measurement system. Moreover, this type of system also allows for the projection, movement and/or alteration of a fixation object or objects from the fixation system without interfering with either of the other systems or their functions. For example, when used in a system for glucose concentration measurement as described above, the use of two different wavelengths for illumination and measurement completely separates and isolates the bleach light source from the sensitive measurement process. Thereby, a sensor that does not respond to the bleaching wavelength does not sense the bleaching light and its output can be amplified for the reflected light at a second wavelength. Additionally, operating the fixation system at a third wavelength selected does to interfere with the operation of the illumination system and also does not produce any additional bleaching. In another alternative embodiment, the fixation light is provided at a wavelength that is the same as one of the other wavelengths but is modulated so that each system remains operable. In another alternative embodiment, the fixation wavelength operates within a narrow wavelength band separate from the band utilized by one of the other systems.

In this embodiment, the illumination system is configured to provide light along an optical pathway to the eye 110, such as for bleaching of the foveal region of the retina to measure blood glucose, as described above. Pulsed light source 140 (e.g., 550 nm) is imaged into the pupil of the eye with sensor/source optics 141 and an eye lens 143. A feedback sensor 145 receives a portion of the light from source 140 via a beamsplitter 144 and may be used for feedback control of the pulsed light source 140. The pulsed source 140 may be filtered by an appropriate interference filter 146 (e.g., an interference filter operating at 550 nm) before passing through a dichroic 156 (which, e.g., transmits wavelengths less than 525 nm and reflects wavelengths greater than 525 nm and another set of optics 157. Thereafter, the filtered light passes through a second dichroic beamsplitter 148 (characterized, e.g., as transmitting wavelengths less than 575 nm and reflecting wavelengths greater than 575 nm), and then travels through the eye lens optics 143 and into the eye 10.

In one particular embodiment, the sensor 150 in the measurement system monitors the portion of the eye acted upon by the illumination system to, for example, synchronize the operation of the measurement system to that of the illumination system.

The measurement system utilizes steady light 151 (at, e.g., 600 nm) for performing, measurement operations in the eye. In one embodiment, the source light 151 is focused at the pupil of the eye to provide light to a broad area of the retina. Reflected light from eye 110 to sensor 150 (such as, e.g., a CCD or photodiode) reflects off dichroic 148, passes through an interference filter 155 (e.g., at 600 nm, to filter out blue light reflected from the eye), sensor/source optics 154 and a beamsplitter 152. Light from light source 151 is reflected through source optics 154 by beam splitter 152. The measurement light is also filtered to a narrow range (e.g., 600 nm) by interference filter 155. In this embodiment, the measurement system light source 151 and the measurement system optics illuminate the eye at significantly different wavelengths to allow for total blocking of the pulsed source light 140 by filter 155. Also, in one embodiment, the light from the source 151 is at an intensity that does not interfere or impede the effective operation of the procedure conducted by the illumination system, such as bleaching for glucose concentration measurement.

In the illustrated embodiment, a data capture system 113 operates in conjunction with the sensor 150. It is to be appreciated that the data capture system could also be used with the other systems separately or an overall data capture system 113 could collect and analyze data from all, some or a combination of the other systems.

In the illustrated embodiment, the sensor 150 is conjugate with the retina of the eye and is thereby in focus with the retina and ideally suited for retinal measurements. The sensor 150 can be, for example, a CCD, CMOS imager, a photodiode or other suitable known optical sensor. Some embodiments of a photodiode can be a more sensitive device than a standard CCD and it can be utilized in the frequency domain to filter out all of the first order effects and only look at the higher order harmonics as described in the above-referenced U.S. Pat. No. 6,650,915, or to make other time-based, frequency-based, or phase-based measurements.

In this exemplary embodiment, a fixation system includes suitable optics to superimpose into the eye/optical line of sight a fixation object. Numerous techniques are available to project the fixation object using light by utilizing known optical components and techniques. In the illustrated exemplary embodiment of the fixation system, a fixation light source 130 projects through an appropriate source lens 131, a shaped aperture 134 in plate 133 into fixation relay optics 132. The fixation light passes through dichroic 156 and optics 157, in order to cooperatively operate, synchronize and not interfere with the other systems. The fixation system may also use other optical components as needed as described herein to project the light from source 130 and the fixation object produced by aperture 134 into the eye. The aperture 134 may have any desired shape to produce the fixation object, such as for example, a geometric shape, irregular geometric shape, a letter, a number, a symbol, a character, of groupings and/or combinations of the above categories.

In another embodiment, the plate 133 could be coupled to a movement device 135 (such as a motor or other device) to induce movement (i.e., lateral, transverse, rotational, periodic, continuous, etc.) of the aperture 134 and the resulting optical image relative to the optical pathway. In this manner, the induced movements of the aperture will, to the patient viewing the system via eye lens 143, appear to move. A moving fixation target may improve the patient's ability gaze to remain fixed during both brief and extended testing, measurement and analysis procedures. Advantageously, the eye movement produced by the eye following the movement of the fixation object in this and other embodiments of the fixation system are such that the desired portion of the eye (i.e., the portion of the eye that is the subject of procedures performed by the illumination and/or measurement systems) remains within the parameters of the systems. For example, in the illustrated embodiment, movement of the fixation object produced by the fixation system would be limited such that eye movement produced to remain fixated on the moving fixation object will remain within the measurement parameters of the sensor 150. The position of the plate 133 relative to the source 130, the type of motor or movement means 135 used and the size, shape, appearance of the aperture 134 may vary from the illustrated embodiment.

In another embodiment, the first light is provided at a wavelength and intensity sufficient to bleach visual pigment. The first light or another light is then used to measure the regeneration of the visual pigment. In one embodiment, the reflectance or the rate of change of reflectance of a portion of the eye (such as the foveal region) is measured. While the bleaching is being induced and regeneration is being measured, a second light is provided into the eye at a wavelength and intensity such that the second light remains visible during the bleaching sequence. The second light may be altered using any one or combination of the above described altering techniques. The second wavelength may be at the same wavelength as the bleaching light, or optionally selected at a wavelength outside the bandwidth used to bleach the desired visual pigment. The second light may be at a wavelength outside of the wavelength used for the bleach and remain perceptible to the eye. In yet another alternative embodiment, the second light may be at a wavelength outside the wavelength used for the bleach of one visual pigment and remain perceptible to the eye, since it is sensed by another, unbleached visual pigment. In one specific embodiment, if the bleaching light were at, for example, 593 nm, the blue cone visual pigment would remain unbleached, and a blue light of approximately 480 nm would remain visible even if the green and red pigments were heavily bleached.

It is to be appreciated that movement of the fixation object may be provided in a number of ways. For example, an aperture adjacent the fixation light source could move. In another example, an aperture, shaped aperture, plurality of apertures or images may be positioned on an off-center disc that rotates in the light produced by the fixation light. The degree of off center position and speed of rotation could be utilized to induce a pattern of movement to help fix the gaze. These and other types of apertures and techniques known to those of ordinary skill may be used to introduce movement of the fixation object when viewed by a patient. In addition, the position of the aperture or other mechanism to induce movement need not be limited to a disc adjacent the fixation light source. Virtually any aperture introduced into the optical pathway may be used to create a non-moving or moving fixation object. Virtually any means of introducing the aperture image onto the optical axis may be used to create a non-moving or moving fixation. For example, through the use of (a) a dichroic; (b) a half mirror; or (c) another suitable structure placed in or projected into the fixation optical pathway.

The manner of the movement of the fixation object may itself useful in maintaining visual fixation. For example, the fixation object can be moved in a pattern that will help maintain visual fixation. The pattern could be regular or irregular. Examples of a regular pattern include fixation object movement to trace the perimeter of a shape such as a rectangle, triangle, polygon, circle, oval or other closed curve. Irregular movement, on the other hand, refers to either the rate of movement of the fixation object or the pattern formed by the movement of the fixation object. An irregular rate of movement could provide that the movement of the fixation object is random, moves and stops or is erratically presented in the field of view. An irregular pattern refers also refers to movement or the rate of movement along the pathway or move along the pathway with pauses or other interruptions in regular progress.

It is also to be appreciated that altering the fixation object could also help a subject remain visually fixated or maintain a fixed gaze. Here, alter can mean a change in the quality of the appearance of the fixation object or the quality or characteristic of the fixation light as perceived by the eye. For example, the frequency and/or intensity of the fixation light may change. In another example, the fixation object may move in and out of focus, appear to grow or shrink in size or zoom, pan, or pull back from the image initially presented.

The fixation object may also be an image, series of images or other fixation objects provided into the optical pathway using a suitable projector. In another embodiment, the projector operation is altered or modified such that the fixation object presented to the patient appears to move. The projector could be used at any suitable point along the optical pathway using known optical components and techniques.

In another aspect, alter refers to a change the appearance of the object itself. The object may, for example, change between different letters randomly or to form words. The object may, for example, represent objects or shapes in some pattern that will encourage the viewer or subject to maintain a fixed gaze in an effort to determine the pattern or predict the next letter, pattern or shape in the sequence.

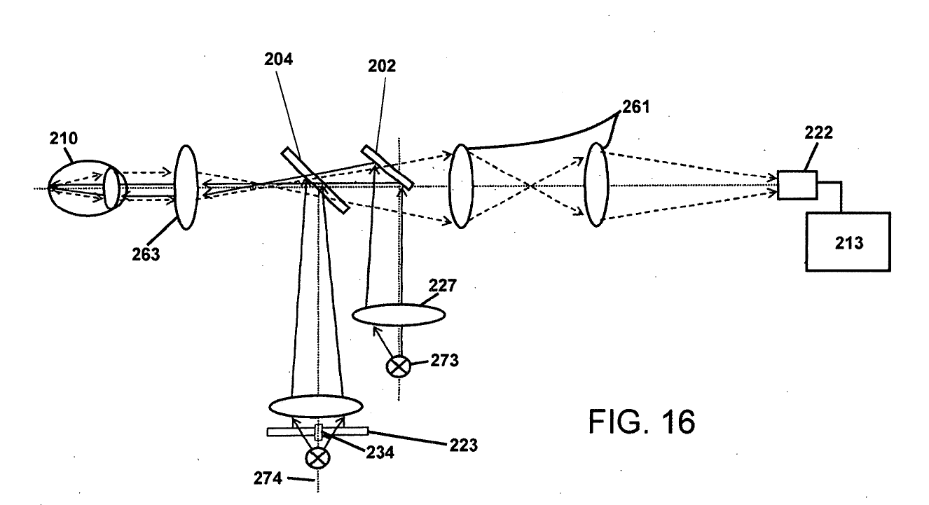

FIG. 16 illustrates another exemplary integrated optical system of the present invention. One notable difference in this embodiment is that the illumination and measurement systems have been combined. Illumination and measurement light is provided by light source 273 (at, e.g., 593 nm) which passes through a 10× microscope objective lens, off of a half mirror 202 and through a dichroic 204 (at, e.g., 550 nm) and an eyepiece 263 to the patient's eye 210. Light reflected from eye 210 passes back through eyepiece 263, dichroic 204 and (in part) through half mirror 202 to a pair of 4× microscope objective lenses 261 to a measurement system, such as an imaging CCD camera 222 and data capture system 213. A region of interest in the eye (e.g., the foveal region) can be selected and imaged based on the experimental requirement. Once selected, the fixation system using a fixation light 274 (operating, e.g., at 480 nm) may be used to fixate the gaze of the eye such that the eye presents a desired orientation to the system. The wavelengths used in lights 273 and 274 may be different, the same and multiplexed or otherwise filtered, or any other combination or selection that allows for the fixation system and the illumination/measurement system to cooperatively operate.

The system is configured such that as a patient looks into the eyepiece 263, the patient is presented with a fixation object generated by the fixation system. As in the previous embodiment, the fixation object is produced by the fixation light 274 passing through a plate 233 with a shaped aperture 234, which passes through dichroic 204 and eyepiece 263 to the patient's eye. As in the previous embodiment, plate 233 may move or be fixed. If movement is used, the movement of the fixation object is constrained to remain within the measurement parameters of the CCD 222 as well as not to interfere with or impede the operation and function of the illumination and measurement system.

Those of ordinary skill will appreciate that the optical component configuration of FIG. 16 is one example of several alternatives that will provide the aspects of the present invention. For example, the plate 233 may be placed elsewhere along the optical pathway used by the fixation light 274. For example, the plate 233 could be positioned at the focal point of the objective lens used in the fixation system.

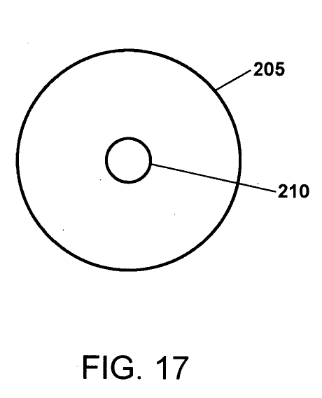

FIG. 17 illustrates the image seen by the patient looking into eyepiece 163. The outer circle 205 represents the viewable region, while the inner circle 210 illustrates the fixation object or image.

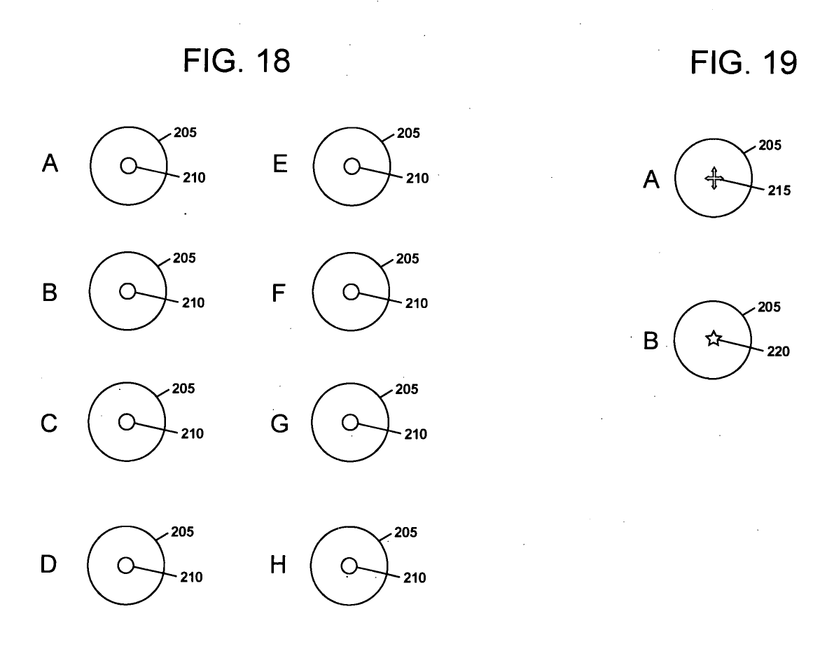

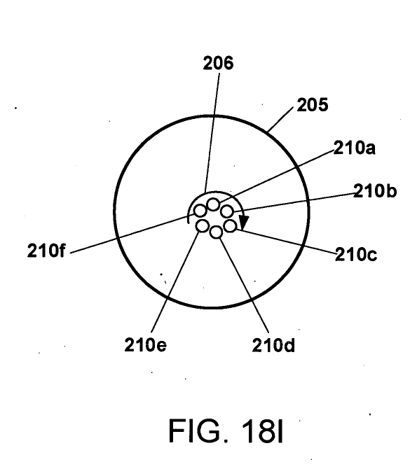

Similar to FIG. 17, FIGS. 18, 19 and 20 illustrate alternative embodiments of the fixation object and different types of motion. FIG. 18 illustrates a series of time elapsed images of a fixation object 210 (e.g., a ring) within a viewable field 205. The various images viewed in order from 18A through 18H are an example of what a patient would see during a procedure where the fixation system projects and moves a fixation object 210. The movement of the fixation object 210 within the field is confined movement near the center of the viewable field 205 in order to accommodate other measurements or data begin collected by other system components. In one example, fixation object movement is restricted to maintain the orientation of the eye presented to the system within the viewable field of the sensors, cameras or other components used by data collection system. FIG. 18I illustrates a time elapsed image of a fixation object 210 as it moves in the direction of arrow 206 through a number of positions 210a-210f within a viewable field 205. The proximity of the various positions 210a-210f also illustrate how a fixation object may move to help maintain fixation yet remain in a confined area such that the fixation object helps the subject fix the gaze in an orientation that remains within the detection or measurement parameters of the system. Alternative fixation objects, cross 215 and star 220, are illustrated in FIGS. 19A and 19B. These and other shapes may be used as fixation objects.

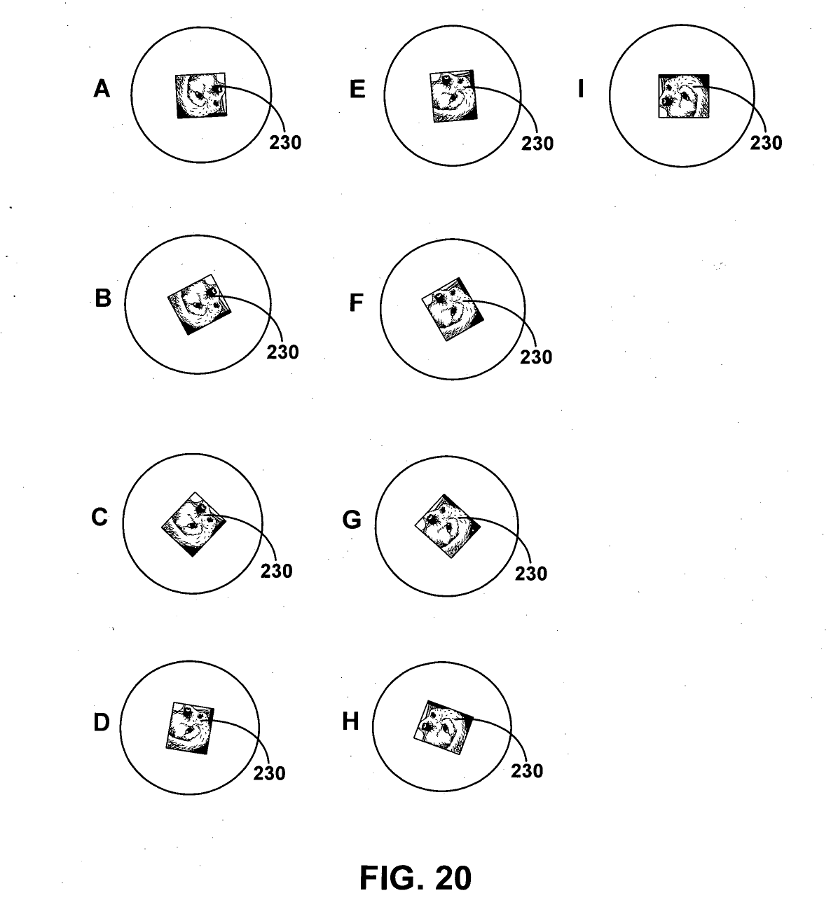

FIG. 20 illustrates a series of elapsed eyepiece images similar to the series presented in FIG. 18. FIG. 20 also provides an example of a fixation object 230 that is an image rather than a shape or object. In addition, the fixation object 230 is presented in an initial orientation within the visual field 205 that may render the image difficult to recognize (FIG. 20A). In this instance, the patient's inability to immediately recognize a familiar image (i.e., image 230 is a dog) may also help remain fixated or maintain a fixed gaze in a desired position. The series of images in FIG. 20 also illustrate another type fixation object movement. As shown by the series of images in FIG. 20A-I, the remains in one position within the visual field 205 and moves by rotating about that point. In alternative embodiments, the image could remain in one orientation (i.e., the orientation of FIG. 20-I) and then move about the visual field 205 as described above with regard to FIGS. 18A-18H. In other embodiments, a plurality of images could be presented (with or without motion) during the procedure to assist the patient in maintaining visual fixation. As discussed above, the degree or amount of movement of a fixation object is related to the visual measurement system limitations. For example, the fixation object may move but maintain the measurement object of interest (i.e., the portion of the eye) within the measurable field of the instrument(s) being used.

While fixation has been described in some embodiments using a light based fixation object, it is to be appreciated that a fixation object may also be provided that utilizes a shadow or darkened area in the visual range. For example, fixation of the gaze could be accomplished by providing an optical system that would block light from a portion of the visual range. The blocked light area may also be formed into shapes, have an altered appearance, as well as move and/or rotate as discussed herein. For example, a circular fixation object could be provided using a black circle appropriately placed in the optical pathway to as to produce a darkened area. In one exemplary embodiment, the incoming light blocked by the object could be selected from a desired fixation wavelength as discussed herein. In this manner, the incoming wavelength of light that is blocked is selected based on other analysis criteria and/or wavelengths used in the optical system.

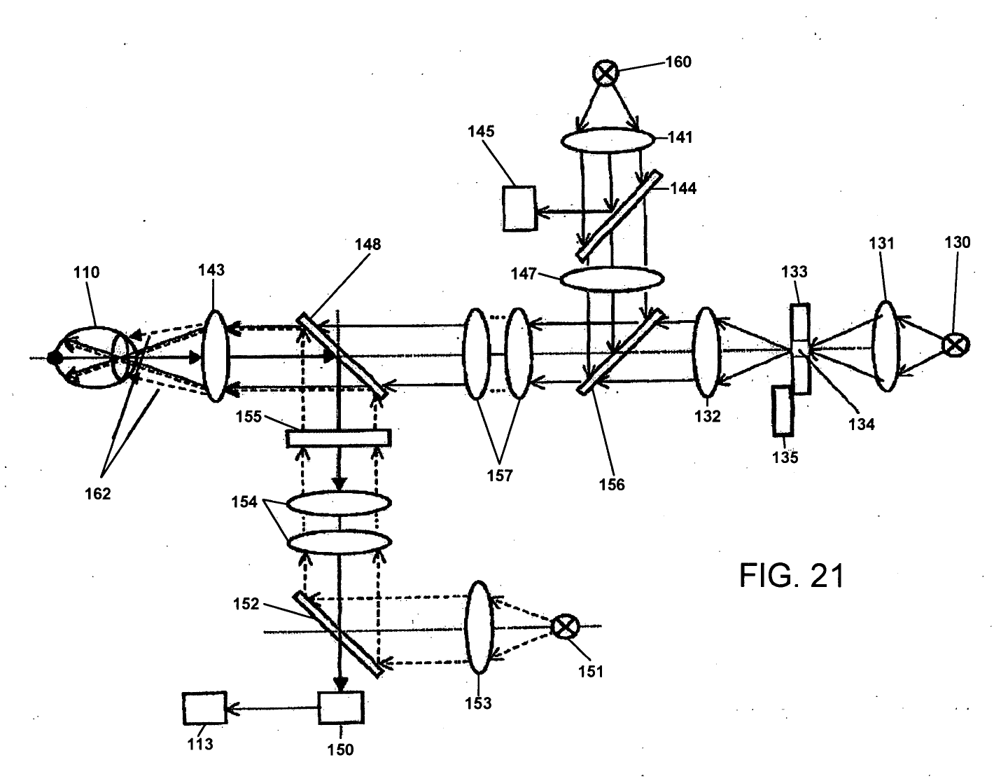

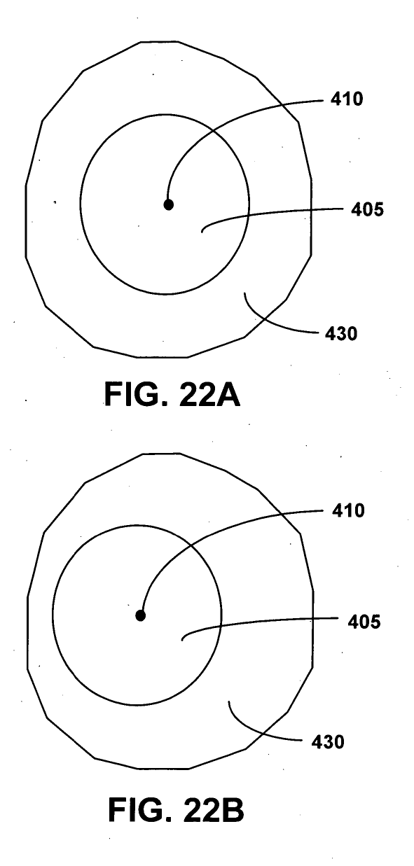

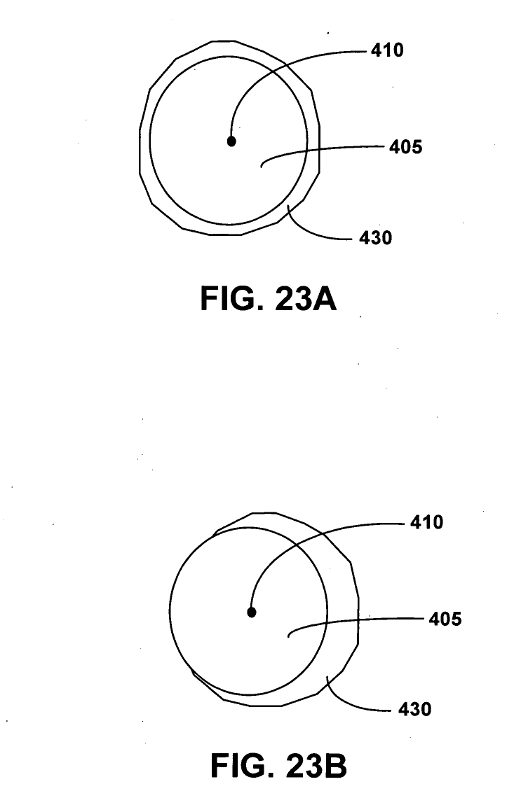

An exemplary system designed to allow the detection and correction of head movement is shown in FIG. 21. This optical arrangement is similar to that shown in FIG. 15, except that the source system is replaced with a surround light or “alignment light” source. The surround light source 160 is preferably a blue LED (or other wavelength that provides reasonable contrast to, or visibility in the presence of, the LED in the illumination system used for regeneration). The light from the LED enters the optical system through shaping lens 147 and dichroic 156, ending up at the front surface of the eye 110 as surround light beam 162. This beam, unlike the beams for illumination and fixation which are small to pass entirely through the pupil, is configured to be larger than the patient's pupil, and provides a roughly circular image of the iris edge 430 as shown in FIGS. 22 and 23. (The iris is not perfectly circular; the appearance of the light beam to the user reflects this shape and is represented as an irregular boundary in those figures.)

The appearance of the overall image as seen by the user is shown in FIGS. 22 and 23. FIG. 22A illustrates the appearance of a system in which the circular image of the illumination beam 405 is approximately concentric with the roughly circular image of the iris 430, indicating proper registration between the eye and the illumination beam. In both images element 410 is a fixation light shown centered in the illumination beam. FIG. 22B shows the appearance after a slight change in relative head position, caused by either a translation or rotation between the beam and the eye. Because the image of the iris moves with the head while the image of the illumination beam and fixation spot remain fixed in the view, the iris image 430 is no longer concentric with the beam image 405. This is an indication to the user to move the head back to the position of proper registration as shown in FIG. 22A.

In FIG. 23A, the appearance of the overall image is shown with a smaller diameter circle 430 for the iris image. This arrangement is advantageous in establishing initial alignment of the instrument to the user, and for re-establishing the correct location when the instrument is again placed on the head for a subsequent measurement of glucose. FIG. 23B again shows the appearance of slight misalignment, providing feedback to the user or technician to restore the head to the correct registration.

Fundus cameras, retinal photography techniques, optics systems and other details are further described in the following references: U.S. Patent Application Publication US2004/0075812 entitled “Device and Method for optical Imaging of Retinal Function” to Kardon et al.; U.S. Pat. No. 6,404,985 entitled “Fundus Camera for Diagnostic Fundus Photography” to Ohtsuka; U.S. Pat. No. 5,315,329 entitled “Simultaneous Stereo Fundus Camera” to McAdams et al. and U.S. Pat. No. 5,820,557 entitled “Blood Glucose Measurement Apparatus” to Hattori et al., each of the above references is incorporated herein by reference in their entirety.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A method for determining blood glucose level in an individual, comprising:
aligning a region of a retina of an eye of the individual with an instrument;
measuring a regeneration rate of a retinal visual pigment of the individual; and
determining the blood glucose level of the individual using the measured regeneration rate of the retinal visual pigment.

2. The method of claim 1 further comprising adjusting an angle between the instrument and the retina region to increase light reflected from the retina region to the instrument.

3. The method of claim 1 wherein the measuring step comprises directing light into the retina region.

4. The method of claim 1 wherein the measuring step comprises measuring a regeneration rate of a retinal visual pigment in the retinal region.

5. The method of claim 4 wherein the retina region comprises a foveal region.

6. The method of claim 1 wherein the aligning step comprises aligning the retina region and instrument for a measurement period of at least 2 seconds.

7. The method of claim 6 wherein the measuring step comprises taking first and second measurements from a portion of the eye with the instrument at first and second points in the measurement period and the determining step comprises using the first and second measurements to calculate a blood glucose concentration.

8. The method of claim 1 further comprising preventing relative movement between the instrument and the eye.

9. The method of claim 8 wherein the preventing step comprises attaching the instrument to a portion of the individual's head.

10. The method of claim 9 wherein the instrument is in the form of a pair of eyeglasses, the attaching step comprising mounting the instrument on the individual's head.

11. The method of claim 8 further comprising calibrating a position of the instrument with respect to the eye.

12. The method of claim 11 wherein the calibrating step comprises adjusting an instrument support element.

13. The method of claim 11 wherein the calibrating step comprises aligning the instrument with an external patient feature.

14. The method of claim 11 wherein the calibrating step comprises aligning the instrument with a retinal feature.

15. The method of claim 1 further comprising providing an indication of a misalignment between the retina region and the instrument.

16. The method of claim 15 where the indication of misalignment between the retina region and the instrument is provided by an alignment light.

17. The method of claim 1 wherein the aligning step comprises directing the individual's gaze toward a fixation target.

18. The method of claim 17 wherein the fixation target comprises an alignment light.

19. An apparatus for glucose measurements comprising:
a blood glucose analysis instrument adapted to determine blood glucose concentration from information regarding regeneration rate of visual pigment in a retina region of an eye; and
an alignment mechanism adapted to align the retina region with at least a portion of the device.

20. The apparatus of claim 19 further comprising an adjustment mechanism adapted to adjust an angle between the instrument and the retina region to increase light reflected from the retina region to the instrument.

21. The apparatus of claim 19 wherein the instrument comprises a light source, the alignment mechanism being adapted to align the retina region with the light source.

22. The apparatus of claim 21 wherein alignment mechanism comprises an alignment light adapted to surround the light source.

23. The apparatus of claim 21 wherein the alignment mechanism comprises a fixation target.

24. The apparatus of claim 23 wherein the fixation target comprises a light source.

25. The apparatus of claim 24 wherein the fixation target light source emits light at a color substantially different than a color of the instrument light source.

26. The apparatus of claim 24 wherein the fixation target light source varies in color or intensity with time.

27. The apparatus of claim 19 further comprising a photodetector, the alignment mechanism being adapted to align the retina region with the photodetector.

28. The apparatus of claim 19 wherein the alignment mechanism is further adapted to provide an indication of misalignment of the instrument and the retina region.

29. The apparatus of claim 19 wherein the alignment mechanism comprises an attachment member adapted to attach the instrument to a portion of the individual's head.

30. The apparatus of claim 29 wherein the instrument and alignment mechanism are configured in a housing in the form of a pair of eyeglasses, the housing comprising the attachment member.

31. The apparatus of claim 19 wherein the alignment mechanism comprises a calibration mechanism adapted to calibrate relative position of the instrument with respect to the eye.

32. The apparatus of claim 31 wherein the calibration mechanism comprises an adjustable instrument support element.

33. The apparatus of claim 31 wherein the calibration mechanism is adapted to align the instrument with a patient external feature.

34. The apparatus of claim 31 wherein the calibration mechanism is adapted to align the instrument with a retina feature.

35. A method of measuring blood glucose concentration of an individual comprising:
maintaining alignment of a retina region of an eye of the patient with respect to a measurement instrument for a measurement period;
taking first and second measurements from a portion of the eye with the measurement instrument at first and second points in the measurement period; and
using the first and second measurements to calculate a blood glucose concentration.

36. The method of claim 35 wherein the measurement period is at least 2 seconds.

37. The method of claim 35 wherein the step of maintaining alignment comprises providing a fixation target and directing the eye's gaze toward the fixation target.

38. The method of claim 35 wherein the step of maintaining alignment comprises preventing relative movement between the individual's head and the instrument.

39. The method of claim 38 wherein the preventing step comprises attaching the instrument to a portion of the individual's head.

40. The method of claim 39 wherein the instrument is in the form of a pair of eyeglasses, the attaching step comprising mounting the instrument on the individual's head.

41. The method of claim 35 further comprising calibrating a relative position between the instrument and the eye.

42. The method of claim 41 wherein the calibrating step comprises adjusting an instrument support element.

43. The method of claim 41 wherein the calibrating step comprises aligning the instrument with a patient external feature.

44. The method of claim 41 wherein the calibrating step comprises aligning the instrument with a retina feature.

45. The method of claim 35 wherein the step of maintaining alignment comprises providing an indication of misalignment of the retina region and the instrument.

46. The method of claim 35 where the step of maintaining alignment comprises directing an alignment light at the eye.

47. The method of claim 35 wherein the steps of taking first and second measurements comprise directing light into the eye.

48. The method of claim 47 wherein the directing step comprises directing light onto the retina region.

49. The method of claim 35 wherein the using step comprises determining a regeneration rate of a retinal visual pigment of the individual.
